(19) United States
(12) Patent Application Publication
(10) Pub. No.: US 2026/0218223 A1
(43) Pub. Date: Jul. 30, 2026

(54) AN ENGINEERED RICE WITH REDUCED VACUOLAR PROTON PYROPHOSPHATASE 5 (VPP5) EXPRESSION

(71) Applicant: THE BOARD OF TRUSTEES OF THE UNIVERSITY OF ARKANSAS, Little Rock, AR (US)

(72) Inventors: Vibha SRIVASTAVA, Fayetteville, AR (US); Peter James ICALIA GANN, Laoag City (PH)

(21) Appl. No.: 19/151,575

(22) PCT Filed: Jan. 26, 2024
(86) PCT No.: PCT/US2024/013116

§ 371 Date: Jul. 28, 2025

Related U.S. Application Data
(60) Provisional application No. 63/441,592, filed on Jan. 27, 2023.

(51) Int. Cl. C12N 15/82; C12N 9/22; C12N 15/11

(52) U.S. Cl. CPC C12N 15/8261; C12N 9/226; C12N 15/111; C12N 15/8213; C12N 2310/20

(57) ABSTRACT

A genetically engineered Oryza sativa seed is disclosed. The seed is genetically engineered so that the RNA expression or protein expression of vacuolar proton pyrophosphatase 5 (VPP5) in the developing seed is reduced by at least 50% as compared to a non-engineered control seed. A method of downregulating VPP5 in a tissue culture of regenerable plant cells or protoplasts is also disclosed.

### CROSS-REFERENCE TO RELATED APPLICATIONS

This application claims priority to U.S. Provisional Patent Application No. 63/441,592, filed Jan. 27, 2023, the entire contents of which are hereby incorporated by reference.

### STATEMENT REGARDING FEDERALLY SPONSORED RESEARCH

This invention was made with government support under grant number 1826836 awarded by the National Science Foundation and under grant number 2017-38821-26412 awarded by the United States Department of Agriculture. The government has certain rights in this invention.

### SEQUENCE LISTING

A Sequence Listing accompanies this application and is submitted as an .xml file named “2023-01-23_169946.00745_WIPO_Sequence_Listing_XML.xml” which is 44,029 bytes in size and was created on Jan. 23, 2024. The sequence listing is electronically submitted and is incorporated herein by reference in its entirety.

### INTRODUCTION

Rice milling yield is both environmentally and genetically controlled (Mckenzie, 1994; Siebenmorgen et al., 2013). High yielding cultivars planted in the southern US and other parts of the world often show low milling quality (Bennett 2012; Fjellstrom et al., 2007; Linscombe, 2009). One attribute of rice grain is ‘chalkiness’ that contributes to milling losses due to grain breakage (Lanning et al., 2011). In addition, grain chalk negatively impacts grain appearance, cooking quality, and palatability (Webb, 1985; Tan et al., 2000). Reducing grain chalk is therefore a major task of southern US rice breeders towards improving rice economy (Fitzgerald et al., 2009; Fjellstrom et al., 2007; Linscombe, 2013).

Chalk is the opaque area in rice endosperm that reduces overall yield by increasing the fraction of broken rice and/or opaque grains. Chalkiness is a complex trait manifested by small, loosely packed starch granules in the endosperm. Numerous chalk quality trait loci (QTLs) have been described in indica and japonica rice (Dwiningsih et al., 2021; Edwards et al., 2017; Sreenivasulu et al., 2015: Zhao et al., 2016), some which are located close to genes such as Waxy, Starch Synthase IIIA, Pyruvate orthophosphate dikinase, UDP glucose pyrophosphatase, cell wall invertase, low phytic acid1-1 (lpal-1), and Chalk5 (Sreenivasulu et al., 2015; Edwards et al., 2017). Of these, Chalk5 QTL was cloned and found to encode vacuolar H+ translocating pyrophosphatase (V-PPase). Higher activity of V-PPase in the indica rice variety, Zhenshan 97 (Z97), contributes to its grain chalkiness, and the V-PPase activity in rice cultivars differing in chalkiness is reportedly due to two cis-elements found in the V-PPase promoter called RY/G-box and CACT tetranucleotide (Li et al., 2014). These two cis-elements are not found in the promoters of the corresponding V-PPase gene in japonica rice. Thus, the genetic elements responsible for chalkiness of japonica rice are still unknown.

### SUMMARY

An engineered Oryza sativa seed or rice is provided herein. The rice may be Oryza sativa subsp. Japonica. The seed is genetically engineered so that RNA expression or protein expression of vacuolar protein pyrophosphatase 5 (VPP5) is reduced by at least 50% as compared to a non-engineered control seed. The genetically engineered seed may include a mutation in the promoter of the VPP5 gene relative to the control seed to reduce the expression of VPP5. The genetically engineered seed may include a mutation in the GATA element located in the promoter of VPP5 relative to the control seed. The genetically engineered rice chalkiness is reduced by at least 50% as compared to rice produced from the control seed.

In a further aspect, a plant or plant part produced by growing the genetically engineered seed is provided.

In a further aspect, a tissue culture of regenerable cells or protoplasts produced from the genetically engineered seed is provided.

In another aspect, a method of producing the genetically modified seed or plant is provided.

In another aspect, a method of identifying low chalk rice lines is provided. The method includes harvesting developing seeds from a rice line, determining the level of VPP5 expression in the developing seeds, and identifying the rice line as a low chalk line based at least partially on the level of VPP5 expression.

### BRIEF DESCRIPTION OF THE DRAWINGS

FIG. 1A is a set of photographs illustrating mature grains from five diverse genotypes matured under the control and high nighttime temperature (HNT). Brilliance was adjusted to −30, shadows to +30 and black point to +100. Lighting was a cool white led light with 120V and the background is a black paper.

FIG. 1B is a table illustrating quantitative analysis of grain chalk and yield under control and HNT conditions. Means within each column (Control and HNT are independent) having the same letter are not significantly different in Tukey's multiple comparison at α=0.05. ** Significant for genotype as source of variation at p≤0.01.

FIG. 1C is a drawing illustrating the function of VPP5 encoding vacuolar H+ translocating pyrophosphorylase (VPPase). V-PPase translocates H+ from the cytoplasm into the vacuole while hydrolyzing inorganic pyrophosphate (PPi) to inorganic phosphate (Pi).

FIGS. 1D-1E are drawings illustrating expression patterns of VPP5 in (D) different tissues and (E) endosperm during grain filling stages indicated by days after fertilization (DAF). Columns for the leaf blade, leaf sheath, root and stem represent 1 and 2 vegetative stage, 3 and 4 represent reproductive stage. Columns in the inflorescence, anther, pistil and lemma/palea are specific sites sampled from the center of each tissue. Columns for the ovary is 1, 3, 5 and 7 DAF. Columns in the embryo are 7, 10, 14, 28 and 42 DAF.

FIG. 2A is a graph illustrating relative expression of VPP5 in the selected genotypes (top to bottom in the legend is shown left to right in the graph) in the grains (caryopses) during early filling stages at 5 to 20 days after fertilization (DAF). VPP5 expression relative to the internal control (7UBQ) is shown. Error bars represent standard error within 3 biological replicates of each genotype/treatment.

FIGS. 2B-2E are graphs illustrating relative expression of VPP5 in ZHE733 (B), Nipponbare (C), LaGrue (D), and Diamond (E) varieties. VPP5 expression relative to the internal control (7UBQ) is shown. Error bars represent standard error within 3 biological replicates of each genotype/treatment.

FIG. 2F are drawings illustrating chromatograms of Sanger sequencing of the target sites in the wild-type (WT) and selected vpp5 lines. Mutations are boxed and described next to each chromatogram. Sequences of target sites are shown above. vpp5-2 target site 2 was sequenced with reverse primer and analyzed by CRISP-ID tool (http://crispid.gbiomed.kuleuven.be/). Reverse complement of the identified mutation is shown below the chromatogram.

FIG. 3A is a schematic diagram illustrating the Nipponbare VPP5 gene showing promoter region (line) and the coding region (arrow). Target sites with their positions relative to the start codon (ATG) are shown. Protospacer adjacent motif (PAM) sequence is underlined and predicted double-strand break (DSB) site is shown (\hat{ }). The promoter elements predicted by Plant PAN 3.0 (Chow et al., 2019) are boxed.

FIG. 3B is a table illustrating a characterization of the selected targeted lines. Mutations are highlighted (gray), deletions are shown by dashed lines and insertions are shown in shaded fonts.

FIGS. 3C-3D are graphs illustrating the relative expression of VPP5 in 10 DAF grains of the targeted (vpp5) and the wild-type (WT) lines and in particular shows an effect of HNT on the expression of VPP5 in the selected vpp5 lines (vpp5-1 and vpp5-2). Error bars represent standard error within three biological replicates of each genotype/treatment.

FIG. 4A is a set of photographs illustrating mature grains from WT and vpp5 lines. Brilliance was adjusted to −30, shadows to +30 and black point to +100. Lighting was a cool white led light with 120V and the background is a black paper.

FIG. 4B is a set of pie charts illustrating a distribution of chalk sizes in control vs. HNT line. Chalk sizes were classified relative to the area of the grain as none (no chalk), small (<10%), medium (11%-20%), and large (>20%). Numbers in the pie chart are frequency counts observed in a sample of 50 grains.

FIG. 4C is a set of micrographs illustrating granule morphology of the translucent and chalky portion of WT and vpp5 grains under scanning electron microscopy (SEM) at 2000× magnification.

FIG. 4D is a set of photographs of mature grains from WT and vpp5 lines. All seeds were harvested in a summer 2021 experiment except vpp5-1-HNT, which is derived from a summer 2022 experiment (See Table 1). Brilliance was adjusted to −30, shadows to +30 and black point to +100. Lighting was a cool white led light with 120V and the background is a black paper.

FIG. 4E is a set of pie charts illustrating the distribution of chalk sizes in each line. Chalk sizes were classified relative to the area of the grain as none (no chalk), small (less than 10%), medium (11%-20%), and large (>20%). Numbers in the pie chart are frequency counts observed in a sample of 50 grains. Grains from summer 2021 experiment were used (see Table 1).

FIG. 5A is a graph illustrating a metabolomic analysis of 15 DAF grains of the vpp5 and wild-type (WT) lines. This figure shows a Principal Component Analysis (PCA) comparing total metabolome of the vpp5-6 lines with WT. Each dot represents an individual sample, red (top) for vpp5-6 and green (lower) for WT (n=6), and surrounding clouds are the 95% confidence intervals.

FIG. 5B is a graph illustrating the top 25 enriched pathways in the 15 DAF grains of vpp5-6. The rich factor is represented by −log 10 (p-value), which is the ratio of differential metabolites in the corresponding pathway to the total number of metabolites identified in the pathway. The larger the value, the higher the enrichment of differential metabolites in the pathway. The color (yellow to red) of the dots represents the P-value of the hypergeometric test. Size of the dots represents the number of differential metabolites in the corresponding pathway (enrichment ratio). PCA and Enrichment analysis was performed in Metaboanalyst (Xia et al., 2015).

FIG. 5C is a graph illustrating Metabolomic analysis of 10 DAF grains of the vpp5 and wild-type (WT) lines. This figure shows the Principal Component Analysis (PCA) comparing total metabolome of the vpp5-6 with the WT. Each dot represents an individual sample, red for vpp5-6 and green for WT (n=6), and surrounding clouds are the 95% confidence intervals.

FIG. 5D is a graph illustrating the top 25 enriched pathways in the 10 DAF grains of vpp5-6. The rich factor is represented by −log 10 (p-value), which is the ratio of differential metabolites in the corresponding pathway to the total number of metabolites identified in the pathway. The larger the value, the higher the enrichment of differential metabolites in the pathway. The color (yellow to red) of the dots represents the P-value of the hypergeometric test. Size of the dots represents the number of differential metabolites in the corresponding pathway (enrichment ratio). PCA and Enrichment analysis was performed in Metaboanalyst (Xia et al., 2015).

FIG. 5E is a graph illustrating metabolomic changes in the grains at 10 DAF and 15 DAF stages, with Principal Component Analysis (PCA) comparing total metabolome of 10 DAF and 15 DAF grains of vpp5-6. Each dot represents an individual sample. The PC variances are shown in brackets. Surrounding clouds are the 95% confidence intervals.

FIG. 5F is a graph illustrating the top 25 enriched pathways in the 10 DAF grains compared to 15 DAF grains in vpp5. The rich factor is represented by −log 10 (p-value), which is the ratio of differential metabolites in the corresponding pathway to the total number of metabolites identified in the pathway. The larger the value, the higher the enrichment of differential metabolites in the pathway. The color (yellow to red) of the dots represents the P-value of the hypergeometric test. Size of the dots represents the number of differential metabolites in the corresponding pathway (enrichment ratio). PCA and Enrichment analysis was performed in Metaboanalyst (Xia et al., 2015).

FIG. 5G is a graph illustrating metabolomic changes in the grains at 10 DAF and 15 DAF stages, with Principal Component Analysis (PCA) comparing total metabolome of 10 DAF and 15 DAF grains of WT (n=6). Each dot represents an individual sample. The PC variances are shown in brackets. Surrounding clouds are the 95% confidence intervals.

FIG. 5H is a graph illustrating the top 25 enriched pathways in the 10 DAF grains compared to 15 DAF grains in WT. The rich factor is represented by −log 10 (p-value), which is the ratio of differential metabolites in the corresponding pathway to the total number of metabolites identified in the pathway. The larger the value, the higher the enrichment of differential metabolites in the pathway. The color (yellow to red) of the dots represents the P-value of the hypergeometric test. Size of the dots represents the number of differential metabolites in the corresponding pathway (enrichment ratio). PCA and Enrichment analysis was performed in Metaboanalyst (Xia et al., 2015).

FIG. 6A is a drawing illustrating the major steps of starch biosynthesis pathway that occur in the cytoplasm with metabolites and the enzymes are shown. SUCSYN, sucrose synthase; UDP-Glc, UDP-Glucose; UGPase, UDP-Glucose Pyrophosphorylase; G1P, Glucose-1-P; AGPase, ADP-Glucose Pyrophosphorylase; ADP-Glc, ADP-Glucose; BT1, Brittle 1 (ADP-Glc transporter); PPi Inorganic Pyrophosphase; Pi, inorganic phosphate. Cellular compartments (vacuole, amyloplast) are indicated.

FIG. 6B is a graph illustrating sucrose concentration in 10 DAF grains of wild-type (WT) and vpp5 lines. Error bars represent standard error of 3 biological replicates consisting of 4 grains each. Significance in Tukey's multiple comparison at α=0.05 is shown by small letters (legend from top to bottom represents left to right in the graph).

FIG. 6C is a graph illustrating starch concentration in mature grains of wild-type (WT) and vpp5 lines. Error bars represent standard error of 3 biological replicates consisting of 4 grains each. Significance in Tukey's multiple comparison at α=0.05 is shown by small letters (legend from top to bottom represents left to right in the graph).

FIG. 6D is a graph illustrating Relative expression of the selected starch biosynthesis genes in 10 DAF grains of WT (left) and vpp5 (right) lines. Standard error within three biological replicates is shown.

FIG. 7A is a graph comparing the expression of VPP5 in KT-6 engineered Kitaake line as compared to the control KW8 line normalized to the rice 7Ubi gene.

FIG. 7B is a graph showing the % chalk in rice grown from KT-6 engineered Kitaake line and the control KW8 line when grown under control or high nighttime temperatures.

FIG. 7C is a set of pie charts illustrating the distribution of chalk sizes in the indicated lines. Chalk sizes were classified relative to the area of the grain as none (no chalk), small (less than 10%), medium (11%-20%), and large (>20%). The charts are frequency counts observed in a sample of 108 grains.

### DETAILED DESCRIPTION

Grain chalkiness is an undesirable trait that reduces milling recovery, eating quality and marketability of rice (Custodio et al, 2019; Webb, 1985). Chalkiness is a genotype-dependent and heat-induced trait manifested by small, loosely packed starch granules in the endosperm that physically appear as chalk-like/white opaque portion in the grain (Ali et al., 2019; Del Rosario et al., 1968; Gann et al., 2021; Lisle et al., 2000; Nevame et al., 2018; Wada et al., 2018). Impeded starch biosynthesis and loading in the rice endosperm is considered as the biochemical basis of grain chalk that contributes to grain breakage during the milling process (Ali et al., 2019; Dhatt et al., 2019; Gann et al., 2021; Lisle et al., 2000; Patindol and Wang, 2003). Rice milling yield is a complex trait. Several high yielding cultivars often show low milling quality in the stress environments, especially high nighttime temperatures (HNT) mainly due to high grain chalk content (Ambardekar et al., 2011; Counce et al., 2005, Lanning et al., 2011; Xu et al., 2020). Reducing HNT effects and preventing grain chalk formation are therefore important goals for the rice breeders (Fitzgerald et al., 2009; Impa et al., 2021).

Starch biosynthesis genes such as Waxy, Starch Synthase IIIA, UDP Glucose Pyrophosphatase, and Cell Wall Invertase influence grain chalkiness (Bao 2014; Sreenivasulu et al, 2015). Perturbation in their expression leads to reduced starch and grain yield (Keeling and Myers, 2010). However, no polymorphism in starch biosynthesis genes has been linked with the natural variation in the chalkiness (Kharabian-Masouleh et al., 2012). On the other hand, numerous QTLs have been reported that control chalkiness (Dwiningsih et al., 2021; Edwards et al., 2017; Gao et al., 2016; Guo et al., 2011; He et al., 1999; Kobayashi et al., 2013; Tabata et al. 2007; Tanet al., 2000; Wan et al. 2005; Zhao et al., 2016). However, only a few causative genes have been identified (Chandran et al., 2022; Li et al., 2014), one of which is Chalk5 that was identified in a population derived from the biparental cross between two indica rice varieties differing in grain chalk content. Chalk5 encodes one of the vacuolar H+ translocating pyrophosphatases (V-PPase). Higher activity of V-PPase in the indica rice variety, Zhenshan 97 (ZH97), contributes to grain chalkiness, and its elevated activity is due to two cis-elements found in its promoter called RY/G box and CACT tetranucleotide (Li et al., 2014). V-PPase maintains pH homeostasis in the cell by translocating H+ ions from the endomembrane system to the vacuole, while hydrolyzing inorganic pyrophosphate (PPᵢ) to inorganic phosphate (Pᵢ) (Ferjani et al., 2011; & Nyrén, et al., 1991). The mechanism of V-PPase hyperactivity derived chalkiness is not clear but Li et al. (2014) suggested that disturbances in the pH of the endomembrane system in the developing endosperm creates airspaces among starch granules, leading to chalky grains. Despite the identification of the favorable Chalk5 allele, breeding for low chalk varieties is complicated by its tight linkage with yield QTLs (Li et al., 2014). Additionally, natural variation in the japonica Chalk5 allele has not been reported, so far.

In our preliminary investigation, we located RY/G and CACT elements in the V-PPase promoter of two more chalky indica varieties, Teqing and ZHE 733. As predicted, these elements were absent in the japonica varieties, including Nipponbare and the elite japonica cultivars (unpublished data). These varieties show lower grain chalk compared to the chalky indica varieties such as ZH97, Teqing, and ZHE 733 (Gann et al., 2021; Esguerra et al., 2021). In a study based on the crosses between non-chalky japonica (Lemont) and the chalky indica rice (Teqing), introgression of the japonica V-PPase allele into indica background resulted in a decrease in grain chalkiness (Zhao et al., 2016). These investigations confirm the role of Chalk5 in the grain chalkiness of indica rice. However, most elite japonica cultivars also develop significant levels of grain chalk and show increased chalkiness under heat stress, especially under HNT (Esguerra et al., 2020; Lanning et al., 2011). Thus, development of high yielding cultivars for rice production in the changing climate is a major challenge towards minimizing milling losses and narrowing the gap between production and the demand for rice as a food crop.

In this present invention, we identified the Chalk5 homolog encoding V-PPase in the japonica rice, evaluated its expression in the developing grains, and targeted its promoter using CRISPR/Cas9. The targeted mutation disrupted a GATA element, leading to the downregulation of V-PPase in the early grain filling stages. Consequently, targeted lines developed more translucent grains that showed significantly reduced chalk in the grains matured in the control or HNT environments. Overall, this study demonstrates that downregulation of V-PPase by editing its promoter elements is effective in reducing grain chalkiness in japonica rice and elucidates a method of improving milling yield of rice cultivated under warmer climatic conditions.

Thus, genetically engineered Oryza plant bodies (e.g., seed, plant, plant part, tissue culture of regenerable cells or protoplasts, or callus), methods of producing genetically engineered Oryza plant bodies, and methods of identifying low chalk rice lines are provided herein. The genetically engineered Oryza plant bodies may include any species of Oryza including but not limited to Oryza sativa and Oryza glaberrima. The genetically engineered Oryza sativa plant body may include any strain/variety of Oryza sativa including but not limited to Japonica (sinica) varieties and may include cultivars such as Nipponbare, Taggart, LaGrue, Kitaake, and Diamond as well as indica varieties such as ZHE 733, ZH97, and Teqing. A tissue culture of regenerable cells or protoplasts refers to an in vitro culture of plant cells or protoplasts capable of forming into a mature, fertile plant.

A plant includes any portion of the plant including but not limited to a whole plant, a portion or part of a plant such as a root, leaf, stem, seed, pod, flower, cell, tissue or plant germplasm or any progeny thereof. Plants also include transgenic plants and non-genetically modified plants. A genetically engineered plant may be engineered using a gene editing system such as a CRISPR/Cas based system or may include more traditional transgenic modification of a plant (e.g., gene guns, electroporation, microinjection, and agrobacterium-mediated transformation). Rice plant refers to whole rice plant or portions or parts thereof including, but not limited to, plant cells, plant protoplasts, plant seeds, plant tissue culture cells or calli. A plant cell refers to cells harvested or derived from any portion of the plant or plant tissue culture cells or calli. Plant parts, include but are not limited to stems, roots, ovules, stamens, leaves, embryos, meristematic regions, callus tissue, gametophytes, sporophytes, pollen, microspores, and the like.

In an aspect, the chalkiness of rice produced from growing the genetically engineered Oryza sativa seeds is reduced by at least 10%, 15%, 20%, 25%, 30%, 35%, 40%, 45%, 50%, 55%, 60%, 65%, 70%, 75%, 80%, 85%, or at least 90% as compared to rice produced from the control seed. This reduction in chalkiness may occur under normal growing conditions or during non-normal growing conditions. For example, the reduction of chalkiness may occur under high nighttime temperature (HNT) growing conditions. HNT growing conditions include temperatures that are higher than normal (e.g., ambient) nighttime temperatures which include but are not limited to approximately 1.0, 1.3, 1.5, 1.7, 2.0, 2.3, 2.5, 2.7, 3.0, 3.3, 3.5, 3.7, or 4.0° C. over normal nighttime temperatures. For example, the HNT may include temperatures greater than 1.0, 1.3, 1.5, 1.7, 2.0, 2.3, 2.5, 2.7, 3.0, 3.3, 3.5, 3.7, or 4.0° C. over normal nighttime temperatures. In Arkansas normal nighttime temperatures are 22° C., but this temperature will vary by location. In the Examples the high nighttime temperatures used were 28° C., but any temperature above 25° C. is considered a high nighttime temperature for the purposes herein. Growth under HNT may also be exemplified by growing the plants under conditions in which the crop plants are exposed to nighttime temperatures of 25° C. or above, suitably 26° C. or above, suitably 27° C. or above, suitably 28° C. or above or even 29° C. as the minimal nighttime temperatures. Chalkiness can be measured using a WinSEEDLE™ Analysis System or using any other instruments or methods known to a skilled artisan.

The non-engineered control seed or simply control seed is defined as a seed genetically identical or nearly-genetically identical to the genetically engineered Oryza sativa seed but does not include the specific genetically engineered mutation that reduces chalkiness in the genetically engineered Oryza sativa seed. It should be noted that the control seed may also have been genetically engineered. For example, the control seed, and thereby the genetically engineered Oryza sativa seed, may already be genetically modified to include an herbicide resistance gene. In another example, the control seed, and thereby the genetically engineered Oryza sativa seed, may have already been genetically modified to include a gene or gene modification (other than the VPP5 alteration) that reduces chalkiness (e.g., a gene or gene modification other than the gene or gene modification included in this disclosure).

The mRNA or protein expression of vacuolar proton pyrophosphatase 5 (VPP5) by the genetically engineered Oryza sativa plant or plant part is reduced by at least 10%, 15%, 20%, 25%, 30%, 35%, 40%, 45%, 50%, 55%, 60%, 65%, 70%, 75%, 80%, 85%, or at least 90% as compared to rice produced from the control seed. The gene sequences in Nipponbare and Kitaake and protein sequence of VPP5 are provided herein as SEQ ID NO: 29, SEQ ID NO: 30 and SEQ ID NO. 31, respectively. The reduction of VPP5 expression is linked to the reduction of chalkiness, as described herein. In some cases, the VPP5 gene is removed or mutated such that the genetically engineered Oryza sativa plant body does not express VPP5 protein, resulting in a 100% reduction in expression (e.g., zero expression). The mRNA or protein expression of VPP5 can be measured and compared in different tissues obtained from the plant, but as the inventors noted, the developing rice seed or grain after fertilization at 5 to 20 days after fertilization is a point at which the expression of VPP5 can be measured. Those of skill in the art are aware of multiple techniques to measure either mRNA or protein expression including via Northern blotting, in situ hybridization, or quantitative reverse transcription-polymerase chain reaction (RT qPCR) for mRNA expression and use of antibodies via immunofluorescence, proximity-based immunoassay with qPCR signal amplification, western blotting or other means to measure VPP5 protein expression.

In an aspect, the genetically engineered Oryza sativa plant body includes at least one mutation in the promoter of the VPP5 gene. The term “promoter” as used here refers to an expression control sequence composed of a region of DNA generally upstream from the site where transcription is initiated. The mutation may include one or more deletions, one or more insertions, one or more substitutions, or one or more translocations. For example, the mutation in the genetically engineered Oryza sativa plant body, may include a removal of three consecutive nucleotides. In another example, the mutation may include the insertion of a single nucleotide. In another example, the mutation may include both the removal of nucleotides at a first position in the VPP5 promoter, and the addition of one or more nucleotides at a second position in the VPP5 promoter.

In an aspect, the genetically engineered Oryza sativa includes a mutation in a GATA element located in the promoter of VPP5 relative to the control (e.g., control seed, control plant, control plant part, control tissue culture of regenerable cells or protoplasts, or control callus). GATA elements are cis-regulatory elements involved in regulation of gene expression. The GATA element may include the nucleotide sequence, GATA, as well as other GATA-like sequences such as the nucleotide sequence, GATC. Mutations that alter the GATA element to a non cis-regulatory element are generally predicted to decrease expression of the associated gene. The mutation of the GATA element may include any type of mutation as described above. For example, the mutation in the GATA element may include the deletion of nucleotides of the GATA sequence (e.g., deletion of three or more nucleotides such as ATC in the GATA element). In the examples, the genetically engineered Oryza sativa plant includes a 3-nucleotide deletion in the GATA element located near position-729 of the VPP5 promoter (e.g., relative to the translation start site for VPP5). For instance, the mutation may include one, two, or three nucleotides of the GATA element. In some aspects, where the promoter of VPP5 included more than one GATA element, more than one GATA element is mutated. In another example, a mutation in the VPP5 region is made between positions selected from −705 and −735, −710 and −730, −740 and −710, −750 and −690, −800 and −650, −900 and −300, or −1000 and +1 of the translation start site for VPP5. In some embodiments, the mutation is within the VPP5 coding region or includes a change to a RY/G box cis-element.

A method for downregulating VPP5 in an Oryza sativa strain or variety, such as a japonica variety, is disclosed. Downregulating includes decreasing VPP5 mRNA or protein expression. The method includes providing an Oryza sativa plant body (e.g., a seed, cell, callus). Providing a seed may include harvesting the seed from a Oryza sativa plant or receiving a seeds from a commercial provider. The method may include inducing the seed or other plant part to form a callus (e.g., a growing mass of unorganized plant cells, such as parenchyma cells). The callus may be formed by any means including but not limited to the application of auxin and cytokinin. The method includes introducing a targeting nucleic acid into the callus or cells of the plant. The targeting nucleic acid may be introduced by any means including but limited to transfection, lipofection, electroporation, nucleofection, particle bombardment, or Argobacterium-based transformation. For example, the targeting nucleic acid may be introduced by generating an Agrobacterium with the targeting nucleic acid subcloned into a tumor-inducing (Ti) or rhizogenic (Ri) plasmid that resides in the Argobacterium. The Argobacterium, or residing plasmids, may also include genes encoding for nucleases including but not limited to Cas9.

The targeting nucleic acid may include any nucleic acid that, when implemented, results in the reduction of VPP5 (e.g., mRNA or protein) or VPP5 activity as compared to a native VPP5 control. The targeting nucleic acid may have a sequence complementary to a target VPP5 sequence. For example, the targeting nucleic may target the VPP5 promoter, a region of the VPP5 promoter, or a VPP5 cis-regulatory element (e.g., a GATA element). In another example, the targeting nucleic acid may target the VPP5 coding region (e.g., to replace or mutate native VPP5 with a less effective or otherwise altered VPP5). In another example, the targeting nucleic acid is a guide RNA (gRNA) that is utilized by nucleases such as Cas9. This is demonstrated in the Examples, but those of skill in the art will recognize that other targeting nucleic acids (gRNAs) distinct from the particular gRNA used herein could be designed to effect a similar purpose of removing the GATA element from the promoter of the VPP5 gene and resulting in reduced VPP5 expression, especially in the developing seed/grain.

The method for downregulating VPP5 further includes introducing a nuclease into the callus, wherein the nuclease causes a change in the nucleic acid sequence of the gene encoding VPP5 or the transcription regulatory region, which may include the promoter or an enhancer or silencer region associated with the gene encoding VPP5 that is targeted by the targeting nucleic acid. The nuclease may include any protein, ribozyme, or riboprotein complex that is capable of cleaving the VPP5 gene including but not limited to a CRISPR (Clustered Regularly Interspaced Short Palindromic Repeats) associated nuclease (e.g., Cas9, Cas12), a zinc-finger nuclease (ZFN), or a transcription activator-like effector nuclease (TALEN). The nuclease may be introduced into the callus as a nuclease-encoded nucleic acid. For example, the nuclease may be introduced as a nuclease-encoded nucleic acid (e.g., RNA or DNA) that is expressed once inside the cells or the callus. For instance, the nuclease may be introduced as a DNA construct-encoding the nuclease which may be subcloned into Ti or Ri plasmid. In another example, the nuclease is introduced as a recombinant protein or combined with the gRNA to generate a ribonucleoprotein complex that may be introduced into the cells or callus by a transfection method such as nucleofection or electroporation.

The method for downregulating VPP5 by generating a genetically engineered plant may include propagating the callus or cells of the plant. The genetic changes in the resulting cells or callus after introduction of the nuclease and the targeting nucleic acid can be tested for incorporation of the genetic change via amplifying the VPP5 promoter region using PCR and sequencing the region of the plant genome in the VPP5 promoter and coding region. Using these techniques, the genetically engineered plant cells comprising the engineered VPP5 can be selected based on sequencing. Propagating the callus may include generating genetically engineered Oryza sativa plants from the cells or callus. In another example, the method includes propagating the callus to produce a genetically engineered Oryza sativa cell culture. The method for downregulating VPP5 may further include selecting the genetically engineered Oryza sativa plants or cells for reduced VPP5 expression. For example, the cells from the genetically engineered plant body (e.g., callus, plant parts) may be isolated and assayed for VPP5 expression (e.g., via PCR, ELISA, western blot, or other method). The plants generated from the cells or callus may also be assessed for decrease VPP5 expression and for decrease chalkiness in the rice produced from the resulting plants.

A method for identifying a low-chalk cell line is also disclosed. In an aspect, the method for identifying a low-chalk cell line includes harvesting seeds or developing seeds from a rice line. The method further includes determining the level of VPP5 expression in the seeds or developing seeds. The expression of VPP5 may be evaluated/determined using any means available to those of skill in the art including but not limited to microarray analysis, rtPCR, real-time quantitative rtPCR, Northern blot analysis, RNA-sequencing, Western blot analysis, or any other means of evaluating the expression of the sequences at either the RNA or protein expression level.

In an aspect, the method further includes identifying the rice line as a low chalk line based the level of VPP5 expression (e.g., relative to control). For example, VPP5 expression levels in the genetically manipulated developing seeds may be compared to VPP5 expression of control seeds, or control developing seeds, that were not introduced to the targeting nucleic acid. The developing seeds may be harvested after fertilization for the purpose of identifying low chalk rice lines by measuring the VPP5 mRNA or protein expression in the lines and comparing to low chalk and high chalk varieties. For example, the developing seeds may be harvested between day 1 and day 30, day 3 and day 25, day 5 and day 20, or day 10 and day 15 after fertilization.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements. The terms “a”, “an” and “the” may mean one or more than one unless specifically delineated.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

The following examples are meant only to be illustrative and are not meant as limitations on the scope of the invention or of the appended claims. All references, including patents, patent publications and non-patent literature, cited herein are hereby incorporated by reference in their entirety. Any conflict between statements in references and those made herein should be resolved in favor of the statements contained herein.

### EXAMPLES

### Example I: Creation of Oryza sativa Line with Reduced VPP5 Expression and Reduced Chalkiness

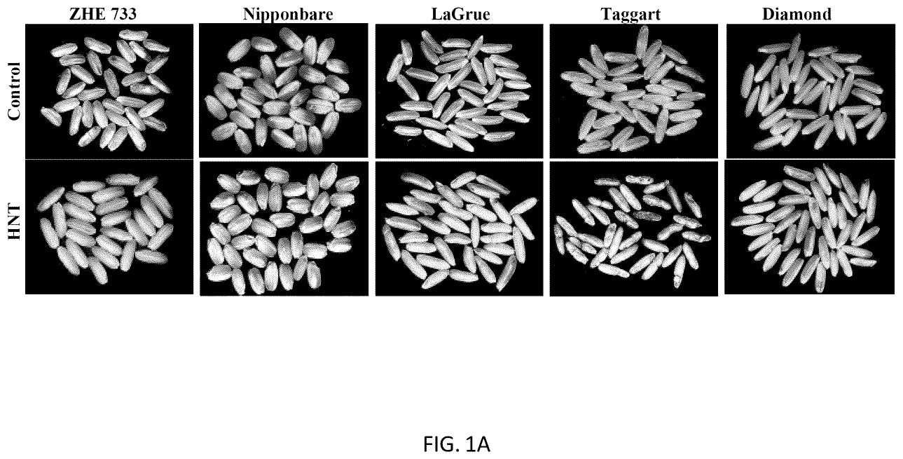

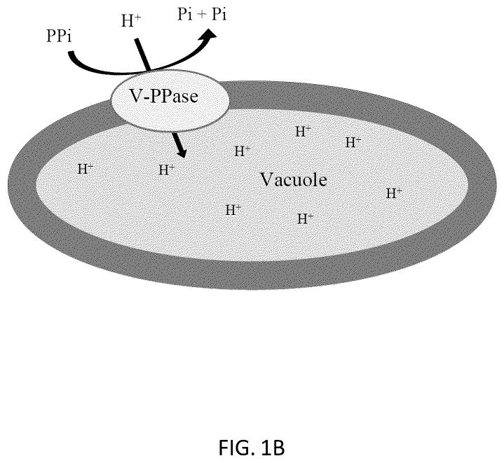

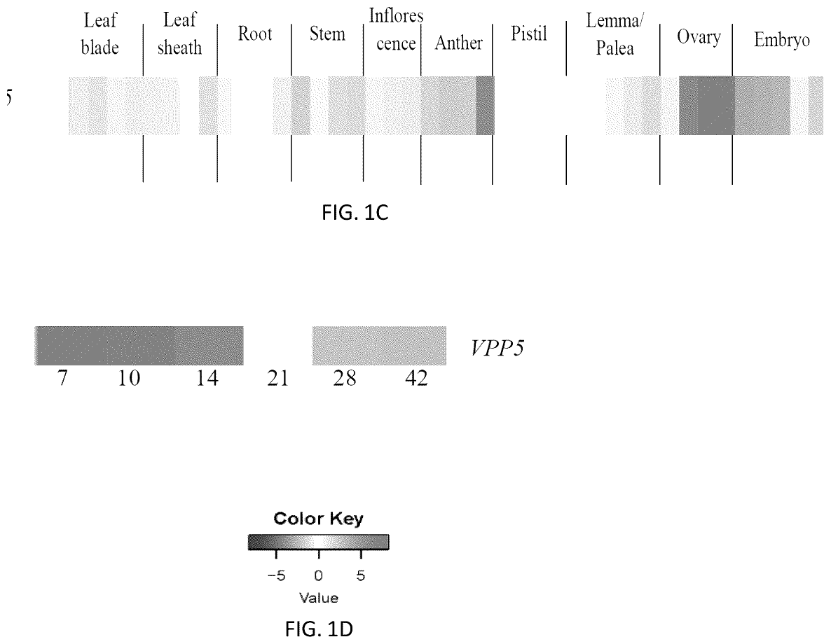

Grain characteristics of different genotypes: The grains of five genotypes were analyzed for the percent area of the grain showing chalk (percent chalk), weight of 1000 grains, and grain filling rate under the control or HNT conditions (Table 1). These include a chalky indica variety, ZHE 733, the japonica variety, Nipponbare, and three tropical japonica elite cultivars, LaGrue, Taggart, and Diamond. As expected, ZHE 733 appeared very chalky (FIG. 1A), which measured 45.67% and 46.10%, respectively, in the control and HNT conditions (Table 1). Among the japonica varieties, Nipponbare appeared chalky, while the elite cultivars were translucent (FIG. 1A). Quantitative chalk analysis of grains matured in the normal condition (control) showed that Nipponbare developed higher percent chalk compared to the elite cultivars. Under HNT, all japonica varieties showed increased chalkiness. Notably, the elite cultivars showed an increase of >3-fold under HNT (Table 1), which is in agreement with the reported chalk increases due to HNT in elite tropical japonica cultivars (Esguerra et al., 2020; Esguerra et al., 2021). LaGrue, Diamond and Taggart developed large size chalk in HNT and appeared very chalky (FIG. 1A). Taggart grains matured under HNT were shrunken, indicating severely impeded starch biosynthesis under heat stress. The grain filling rate was also impacted in each genotype under HNT with Nipponbare and Taggart showing the greatest decline (Table 1). The effect of HNT on the milling yield (head rice yield) has been studied on three of these genotypes, ZHE 733, LaGrue, and Diamond, by Esguerra et al. (2021). This study reported a significantly lower head rice yield for these genotypes under HNT condition, indicating a negative effect of grain chalk on the milling yield. Overall, these genotypes showed variable chalkiness and grain yields and the elite cultivars showed increased chalkiness and lower yield under HNT.

TABLE 1
Quantitative analysis of grain chalk and yield

| | | Chalkiness | Weight of 1000 | Grain Filling |
|---|---|---|---|---|
| | Lines | (%) | Grains (g) | Rate (%) |
| Control | | ** | ** | ** |
| | ZHE 733 | 45.67^{a} | 29.70^{a} | 78.52^{b} |
| | Nipponbare | 12.93^{b} | 17.60^{d} | 85.19^{a} |
| | LaGrue | 8.45^{c} | 27.80^{b} | 59.06^{d} |
| | Taggart | 9.14^{c} | 22.71^{c} | 56.80^{d} |
| | Diamond | 4.80^{d} | 27.90^{b} | 65.56^{c} |
| HNT | | ** | ** | ** |
| | ZHE 733 | 46.10^{a} | 28.30^{a} | 55.06^{a} |
| | Nipponbare | 18.36^{d} | 15.30^{c} | 19.64^{c} |
| | LaGrue | 36.68^{c} | 19.03^{b} | 43.60^{b} |
| | Taggart | 41.04^{b} | 10.17^{d} | 11.20^{d} |
| | Diamond | 15.69^{d} | 12.65^{cd} | 35.93^{c} |

Means within each column (Control and HNT are independent) having the same letter are not significantly different in Tukey's multiple comparison at α=0.05. ** Significant for genotype as source of variation at p≤0.01.

Expression pattern of VPP5 in diverse genotypes: To understand the role of V-PPase in the chalkiness of japonica rice, its expression pattern was studied in the early grain filling stages. Rice contains six V-PPase genes, each encoding the membrane-bound proton pump energized by inorganic pyrophosphate (FIG. 1B). The six V-PPase genes are differentially expressed, one of which underlies the major QTL, Chalk5, associated with the grain chalk trait. This V-PPase(Os05g0156900), referred to as VPP5, is expressed at very low levels in most tissues (Liu et al.,2010). The heatmap of spatio-temporal expression of VPP5 showed that it is mainly expressed in the reproductive tissues and the embryos (FIG. 1C), indicating its functional specialization. Further, VPP5 is upregulated in the developing endosperm during 7-14 days after fertilization (DAF), followed by a rapid decline in the subsequent stages (FIG. 1D). Therefore, VPP5 could play an important role in maintaining pH homeostasis in the endosperm during rapid filling stages that are important for the crop yield and the development of translucent rice grains.

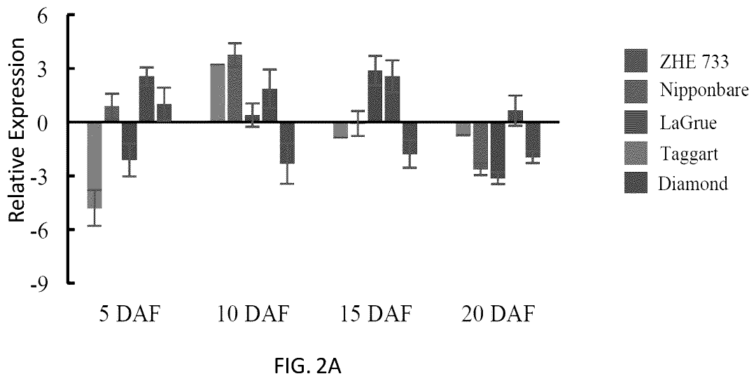

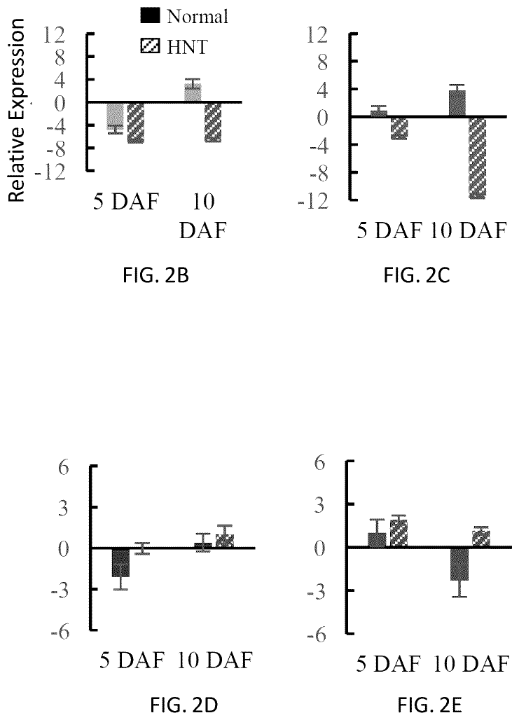

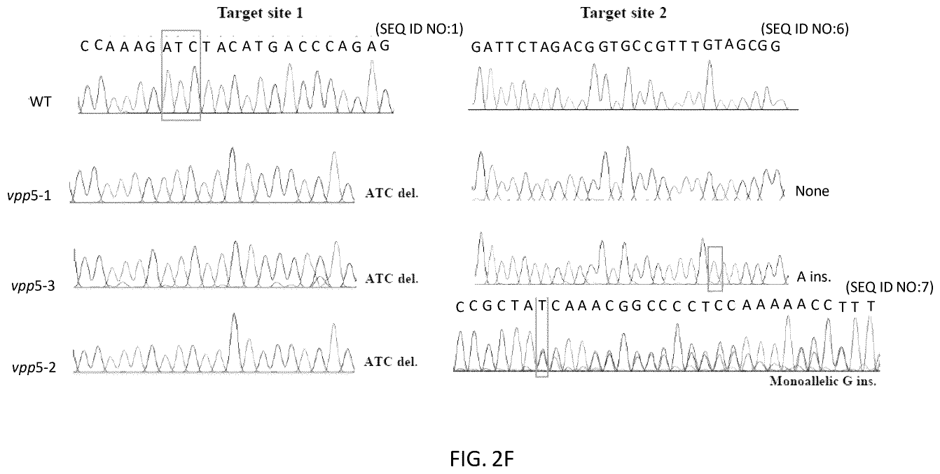

To determine the expression pattern of VPP5 in the genotypes described above, milky/dough grains at 5, 10, 15, and 20 DAF were subjected to gene expression analysis by realtime PCR (FIG. 2). Although, the expression pattern varied between the genotypes, VPP5 was generally upregulated during 5-15 DAF and downregulated at 20 DAF, which is consistent with the heatmap of the expression pattern developed using publicly available datasets (FIG. 1C). The chalky variety, ZHE 733, showed a spike at 10 DAF and a steep decline thereafter. Nipponbare showed a gradual increase from 5 to 10 DAF and a decline thereafter. LaGrue showed a relatively lower expression at 5 and 10 DAF but spiked at 15 DAF. Taggart and Diamond showed opposite patterns. Taggart consistently expressed VPP5 during 5-15 DAF, while Diamond showed consistently low expression during 5-15 DAF (FIG. 2A). The effect of HNT on VPP5 was evaluated in 4 genotypes using the grains at 5 and 10 DAF. Taggart could not be included because of its severely impacted fertility under HNT. This analysis revealed a similar effect of HNT in ZHE 733 and Nipponbare, showing downregulation of VPP5 by HNT and a >10-fold downregulation in 10 DAF grains. LaGrue and Diamond, on the other hand, showed upregulation by HNT, which was greater for LaGrue in 5 DAF grains and for Diamond in 10 DAF grains (FIG. 2B-E). Overall, this analysis showed differential patterns of VPP5 expression and a differential response to HNT among the tested genotypes. The variable expression patterns of VPP5 could account for variable susceptibility to chalkiness in these genotypes under HNT condition.

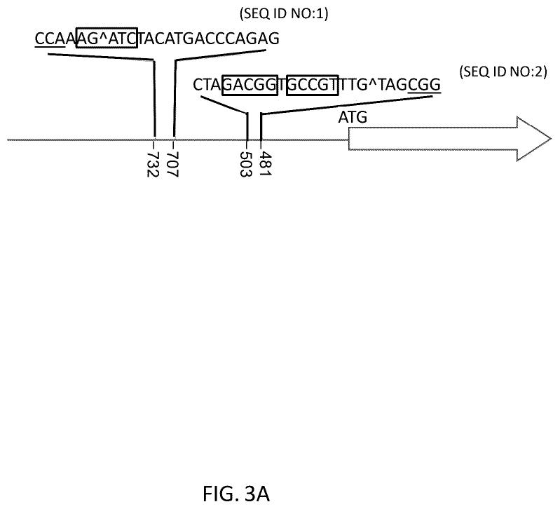

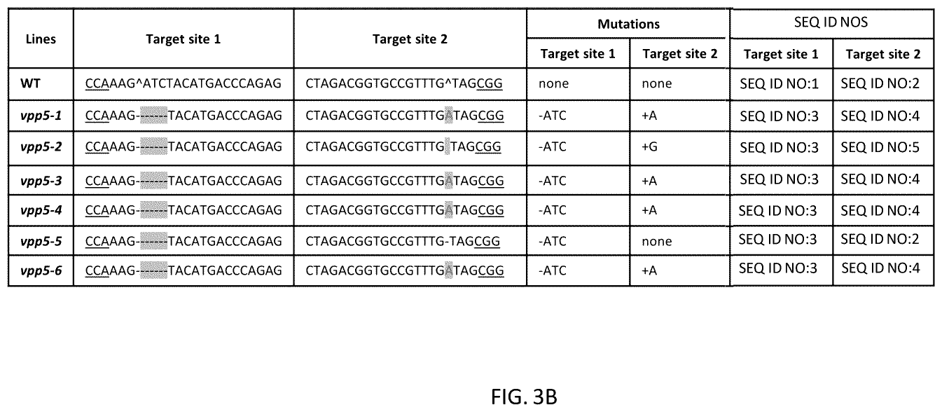

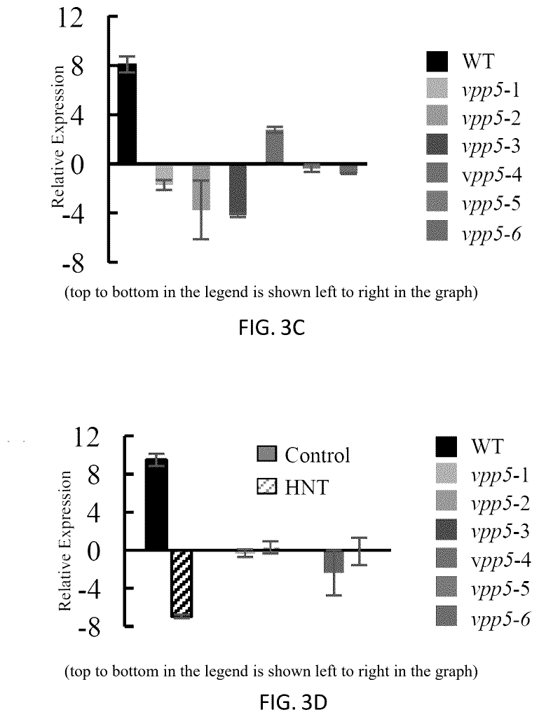

Targeted mutagenesis of VPP5 promoter by CRISPR/Cas9: To determine the role of VPP5 in the grain chalkiness of japonica rice, targeted mutagenesis by CRISPR/Cas9 was carried out in the model japonica rice, Nipponbare. We attempted to create large deletions by simultaneous targeting of two sites located between −732 and −481 in the promoter (FIG. 3A). Transformation of rice with a CRISPR/Cas9 vector containing two sgRNAs, generated 13 independent plant lines, none of which showed fragment deletion on PCR across the target sites. However, 8 of these lines harbored chimeric point-mutations indicated by multiple overlapping peaks in the sequencing chromatogram of the PCR fragment (Data not shown). T1 seedlings derived from 6 of these lines, named vpp5-1 to vpp5-6, were analyzed by PCR followed by Sanger sequencing. This analysis revealed the presence of identical point-mutation (−3) at target site 1 in all 6 lines but variation in the target site 2. These lines were either wild-type (untargeted) at target 2 or carried insertion of A or G (+1) at the predicted double-strand break site (FIG. 3B; FIG. 2F). Although clonal origin of these lines cannot be ruled out, occurrence of identical mutation in the independent events has been reported earlier in nuclease-mediated targeting including CRISPR/Cas9 (Jacobs et al., 2015; Pathak et al., 2019; Zhang et al., 2014). Therefore, we labeled these lines as the independent vpp5 lines.

The promoter analysis of the mutant lines showed that the GATA element consisting of the consensus AGATC sequence was disrupted by −3 mutation at target site 1, whereas no significant change occurred by +1 mutation in target site 2, which contains NAC (GACGG) and dehydrin (GCCGT) elements (FIG. 3A). These lines (homozygous −3) were advanced to T2 generation for further analysis.

Expression of VPP5 in the mutant lines: T2 plants harboring homozygous-ATC mutation in the GATA element were grown in the greenhouse for gene expression analysis. Based on the peak expression of VPP5 in Nipponbare (FIG. 2A), we selected the 10 DAF grains for gene expression analysis. This analysis showed that the vpp5 mutants expressed VPP5 at 5 to 12-fold lower than the wild-type (WT) (FIG. 3C), indicating downregulation of VPP5 upon disruption of the GATA element at −728 to −724 position. Next, VPP5 expression was determined in a subset of 2 vpp5lines (vpp5-1 and vpp5-2) treated with HNT. Plants entering the reproductive stage (collar formation on flag leaf), were transferred to the HNT growth chamber, and the grains collected at 10 DAF were used for the analysis. Although in WT, VPP5 was ~17-fold downregulated by HNT, no significant difference was observed in vpp5 lines (FIG. 3D). In summary, we generated stable mutant lines in the japonica rice background that showed downregulation of VPP5 during early grain filling stages and generally unperturbed expression under HNT. The seeds obtained from these lines were subjected to grain chalk analysis.

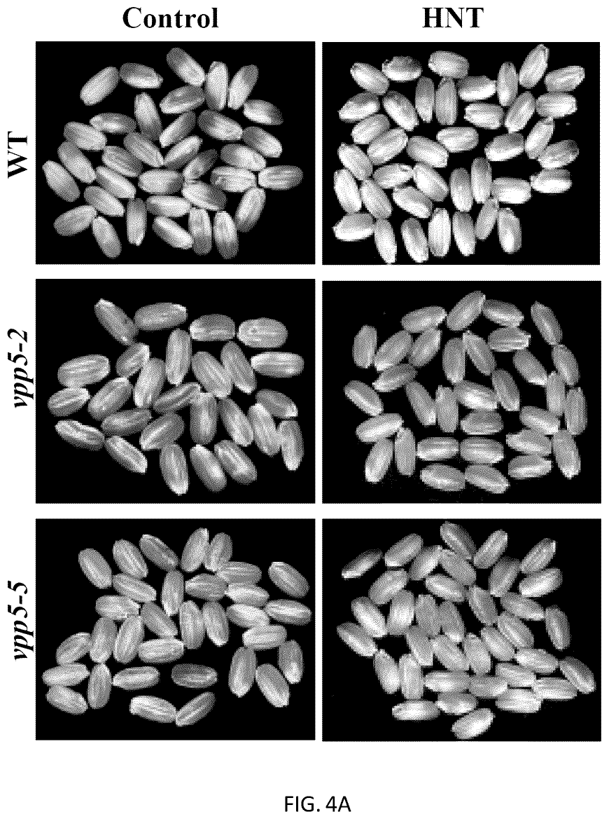

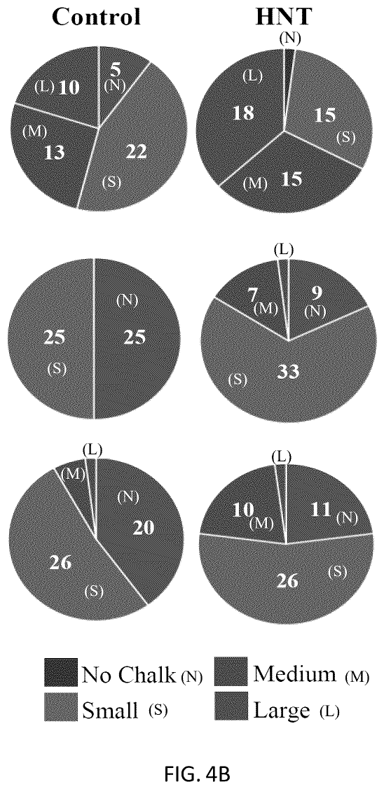

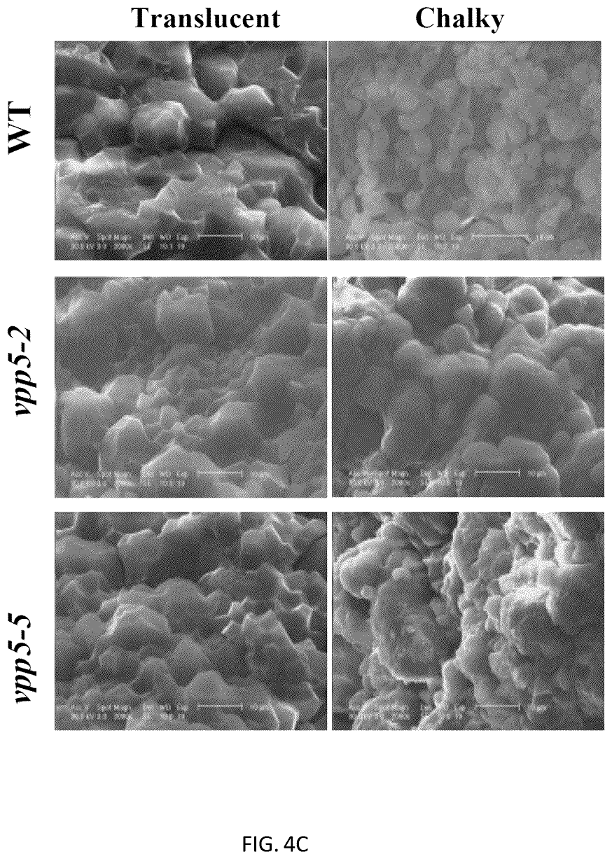

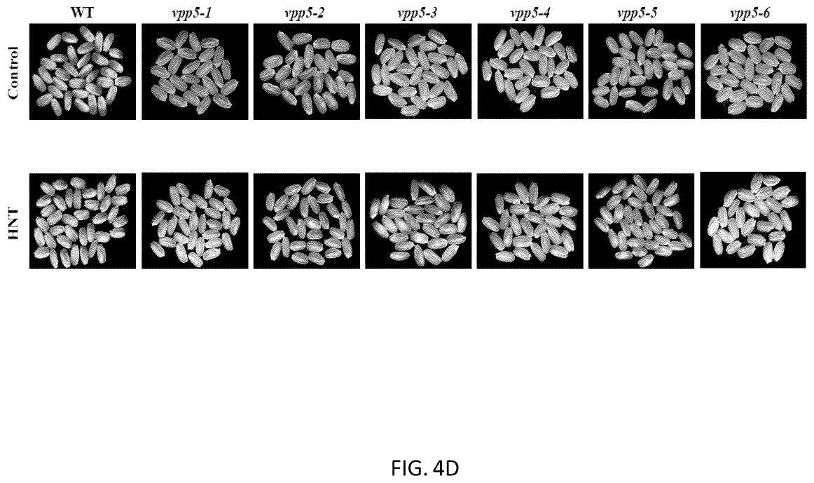

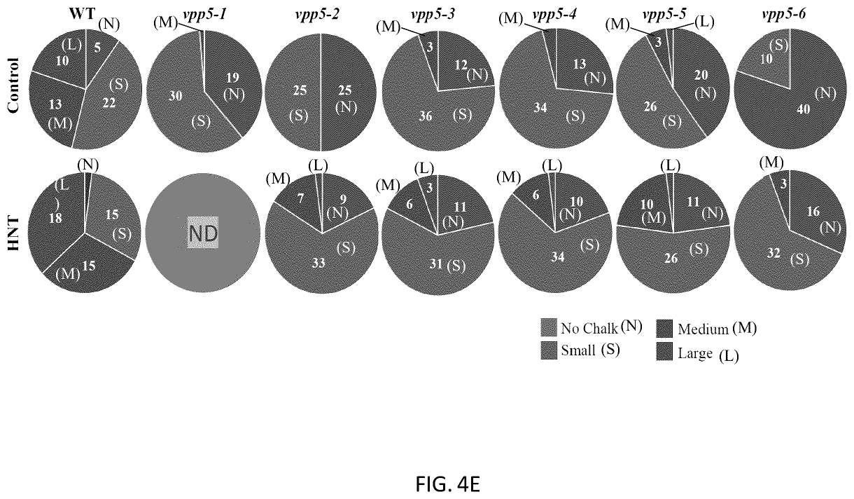

Grain chalk contents in the mutant lines: Grain chalk was analyzed in two separate experiments carried out in the summers of 2021 and 2022 under the control and HNT conditions. The difference in the chalkiness between WT and vpp5 grains could be visualized and captured in a photograph that shows more chalk in the WT grains under control or HNT conditions in a side-by-side comparison with the vpp5 grains (FIG. 4A, FIG. 4D). The quantitative analysis on WinSEEDLE™ showed a significant difference in the percent chalk between the WT and vpp5 grains (Table 2). In the summer 2021, the grain chalk in the WT was 11.95%, whereas in vpp5 lines it varied between 1.04-2.37% (average=1.61) that is 7.38-fold lower (p<0.01) than that of the WT in the control condition. The HNT treatment induced chalk formation in each genotype; however, WT showed significantly higher chalk (17.02%) than the vpp5 lines (2.29-6.05%) (Table 2). On an average, 3.9-fold lower chalk (p<0.01) was observed in vpp5 lines as compared to the WT in HNT. In Summer 2022, a similar trend was observed. Specifically, WT grains developed 9.58% chalk under the control condition, whereas the vpp5 lines showed a range of 0.35-1.06% chalk. In the HNT condition, grain chalk in WT increased to 13.11%, whereas in vpp5 it ranged between 2.54-6.46% (Table 2). The proportion of grains showing small, medium, or large chalk was also different in the WT and vpp5 lines (FIG. 4B; FIG. 4E). In comparison to WT grains, vpp5 lines had a much higher proportion of grains without any measurable chalk. The proportion of grains with small chalk among vpp5 lines varied, but most notably, large chalk was rarely found in the vpp5grains. In HNT, the proportion of WT grains without any measurable chalk sharply declined and the proportion of grains with large chalk increased. Whereas, in vpp5 lines, the increase was mainly in proportion of grains with small or medium chalk, whereas only a very small portion developed large chalk (FIG. 4B; FIG. 4E). Grain weight of vpp5 lines was generally higher than that of that. In summer 2021, grain weight was impacted by HNT treatment in the WT and most of the vpp5 lines; however, in summer 2022, no significant effect of HNT treatment was observed. Grain filling rates were highly variable in vpp5 lines but significant differences between vpp5 and WT were observed except in the experiment conducted in the control condition in summer 2022 (Table 2).

TABLE 2
Grain chalk and yield analysis

| | Summer 2021 | | | Summer 2022 | | |
|---|---|---|---|---|---|---|
| | | | Grain filling | | | Grain filling |
| Lines | % Chalk | Grain Weight | rate | % Chalk | Grain Weight | rate |
| Control | ** | ** | ** | ** | ** | ns |
| WT | 11.95ª | 17.60^{d} | 85.19ª | 9.58ª | 16.68^{b} | 82.35 |
| vpp5-1 | 1.71^{b} | 21.10^{c} | 47.37^{cd} | 0.68^{cd} | 18.64ª | 80.64 |
| vpp5-2 | 1.04^{b} | 24.96^{ab} | 32.20^{ef} | 0.35^{e} | 19.58ª | 79.59 |
| vpp5-3 | 1.29^{b} | 26.60ª | 59.62^{bc} | 0.84^{bc} | 19.82ª | 71.89 |
| vpp5-4 | 2.14^{b} | 21.90^{bc} | 72.55^{ab} | 0.52^{de} | 18.35ª | 78.61 |
| vpp5-5 | 2.37^{b} | 20.90^{c} | 19.05^{f} | 0.56^{cde} | 18.62ª | 61.18 |
| vpp5-6 | 1.16^{b} | 21.45^{c} | 38.00^{de} | 1.06^{b} | 19.62 | 76.60 |
| HNT | ** | ** | ** | ** | ns | ** |
| WT | 17.02ª | 15.30^{c} | 19.64^{c} | 13.11ª | 16.42 | 29.47^{c} |
| vpp5-1 | nd | nd | no | 2.54^{d} | 18.89 | 72.90ª |
| vpp5-2 | 4.56^{b} | 15.40^{c} | 15.52^{c} | 3.68^{d} | 18.73 | 58.10^{b} |
| vpp5-3 | 4.68^{b} | 17.55^{c} | 26.32^{ab} | 4.64^{bc} | 18.50 | 63.64^{ab} |
| vpp5-4 | 4.17^{bc} | 16.90^{c} | 30.56ª | 3.10^{cd} | 17.65 | 66.67^{ab} |
| vpp5-5 | 6.05^{b} | 21.65ª | 20.59^{bc} | nd | nd | nd |
| vpp5-6 | 2.29^{c} | 19.10^{ab} | 15.25^{c} | 6.46^{b} | 18.08 | 69.52^{ab} |

Means within each column (Control and HNT are independent) having the same letter are not significantly different in Tukey's multiple comparison at α = 0.05.
**Significant for line as source of variation at p ≤ 0.01;
ns, non-significant.
nd, not determined;
nd, not determined

Granule morphology of vpp5 lines: The granule morphology analysis by scanning electron microscopy found no difference in the shape, size, and arrangement of the granules in the nonchalky portion of the WT and vpp5 grains (FIG. 4C). As expected, both WT and vpp5 contained compact, large, polyhedral granules, characteristic of translucent grains. However, the chalky grains of these lines matured under HNT showed interesting differences. Although, both WT andvpp5 showed characteristic rounded shaped granules in the chalky portion, the sizes of these rounded granules were different in the WT and vpp5 grains. The WT contained smaller and more loosely packed granules, whereas vpp5 contained both small and large granules (rounded) (FIG. 4C), presumably accommodating smaller size chalk. Overall, granule morphology of the WT andvpp5 lines was indistinguishable but the chalky portion of vpp5 lines generally contained larger rounded granules.

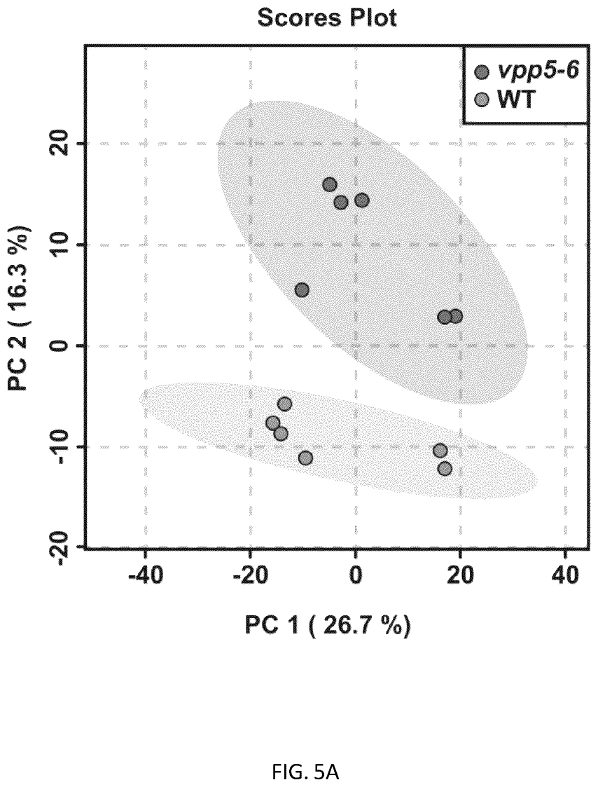

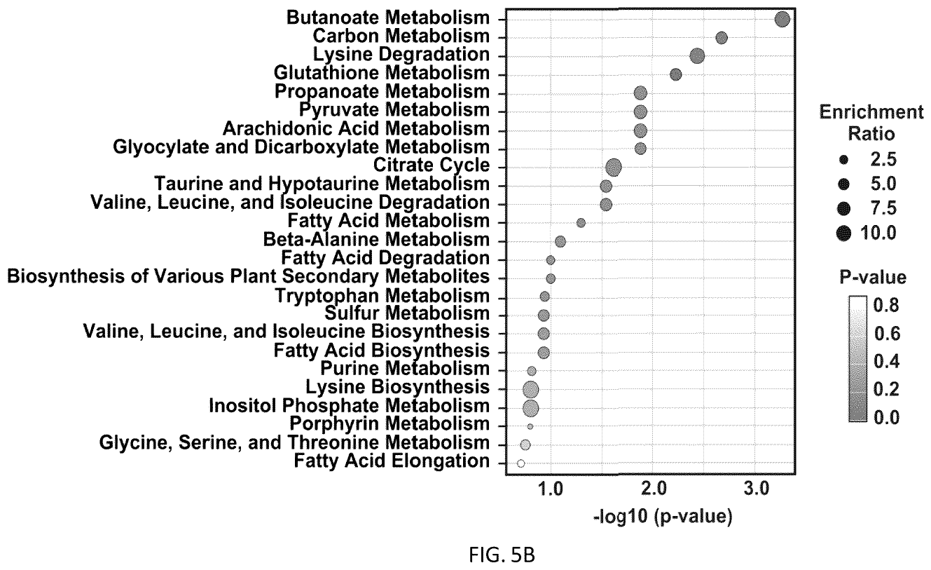

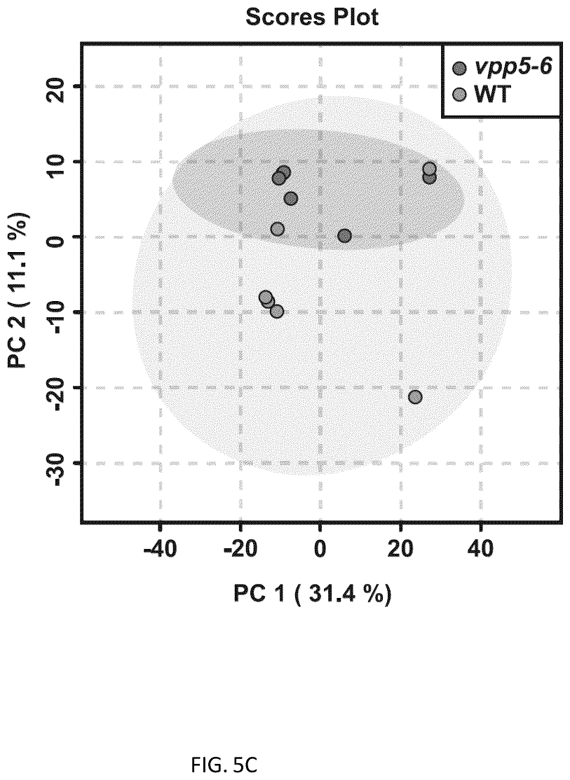

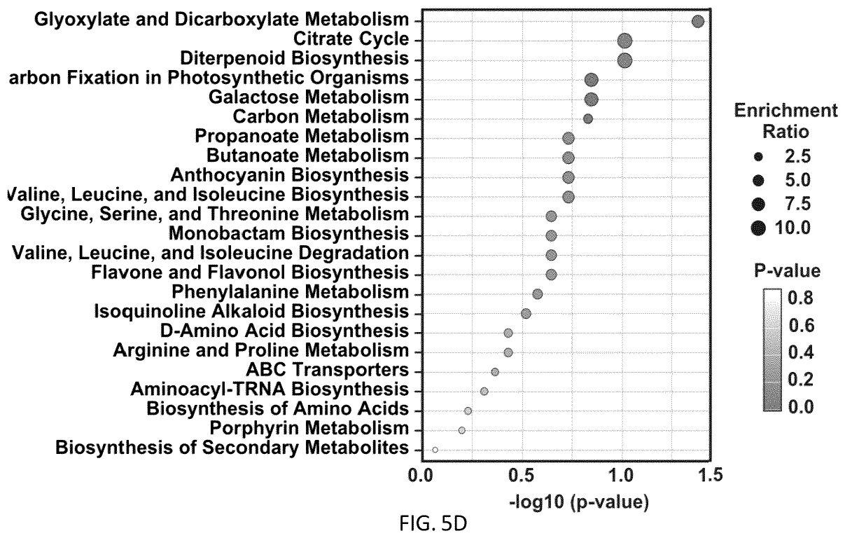

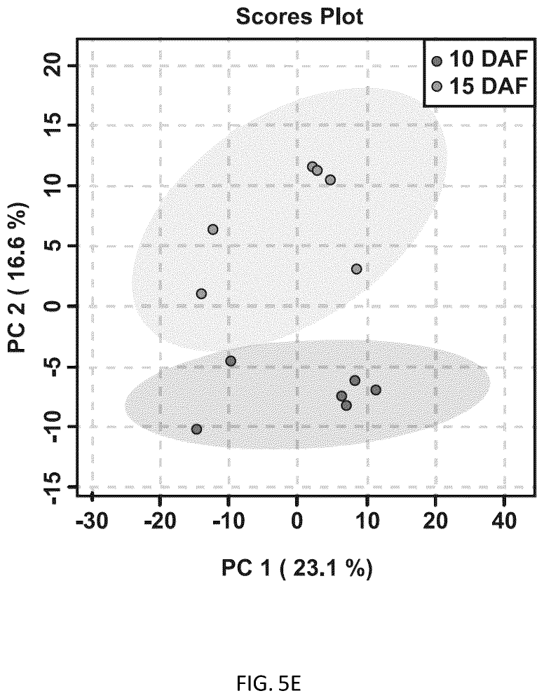

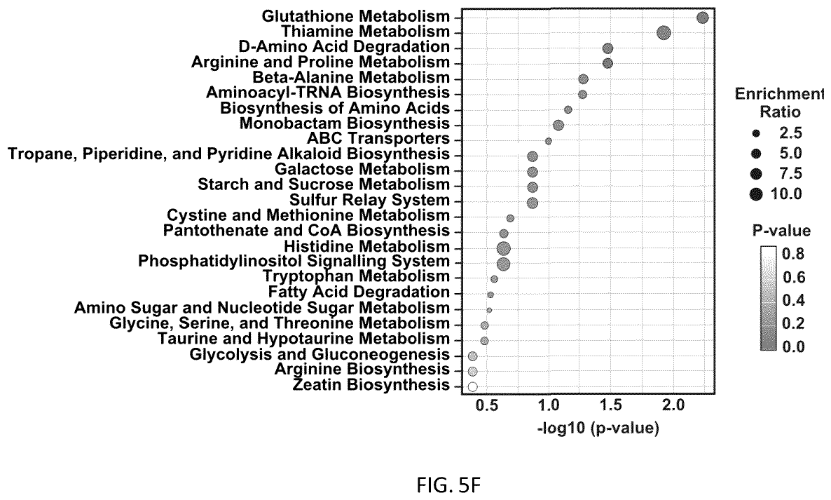

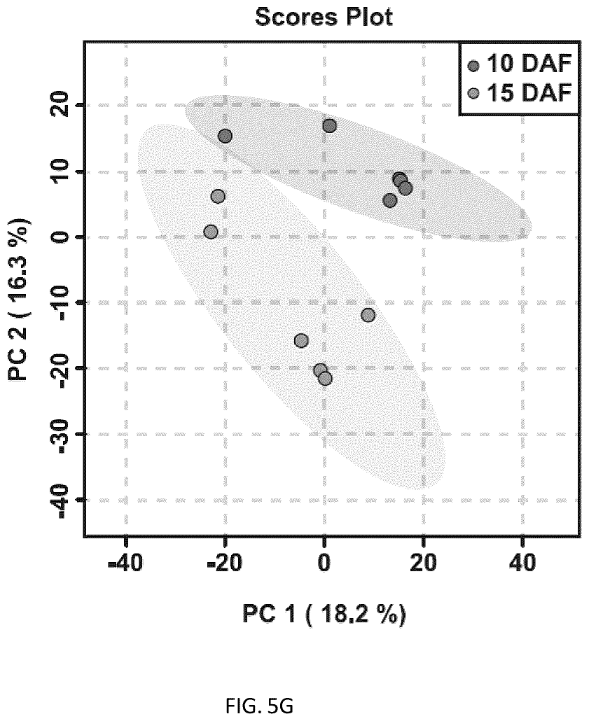

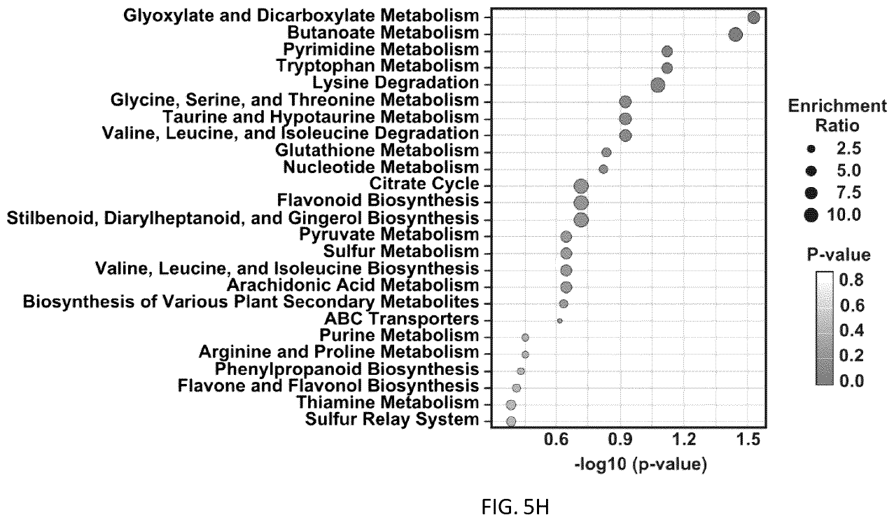

Metabolite Profiling: Grains of the WT and vpp5-6 lines at two developmental stages, 10 DAF and 15 DAF, were subjected to LC-MS for metabolite profiling. This selection was based on the highest expression of VPP5 observed in 10 DAF grains of Nipponbare. Only 19 metabolites in 10 DAF grains were significantly different (p<0.05) in the vpp5-6 mutant as compared to the WT (Table 3). However, in 15 DAF grains, 84 metabolites were significantly up- or down-regulated (p<0.05) in vpp5-6 as compared to the WT (Table 4). Accordingly, no significant difference in the total metabolome of the 10 DAF grains of vpp5-6 and WT was observed in the Principal Component Analysis (FIG. 5C), while a significant difference was observed in the 15 DAF grains (FIG. 5A). As expected, each genotype showed a greater difference in their respective 10 and 15DAF grains, indicating rapid metabolic changes in the grains during early filling stages (FIG. 5E, 5G).

TABLE 3
Differentially regulated metabolites in 10 DAF grains of vpp5-6 vs 10 DAF
grains of WT rice lines.

| KEGG | | | | | | | log2 |
|---|---|---|---|---|---|---|---|
| ID | Compound | m/z^{†} | RT^{‡} | p value | Change | FC^{§} | (FC) |
| C05447 | UDP-glucose | 566.1605 | 5.1 | 3.06E−04 | Up | 3.60 | 1.85 |
| C01144 | (S)-3-Hydroxybutanoyl-CoA | 853.6177 | 37.94 | 9.21E−04 | Down | 0.26 | −1.94 |
| C00672 | Paclitaxel | 853.6857 | 37.53 | 1.24E−03 | Down | 0.48 | −1.07 |
| C17587 | Quercitrin | 448.2627 | 23.11 | 3.26E−03 | Down | 0.32 | −1.63 |
| C02335 | Lipid X | 711.6454 | 21.09 | 3.70E−03 | Up | 2.04 | 1.03 |
| C00016 | Tubocurarine | 609.1959 | 5.19 | 6.05E−03 | Up | 2.86 | 1.51 |
| C07394 | 10-Formyltetrahydrofolate | 473.4873 | 35.6 | 7.69E−03 | Up | 2.25 | 1.17 |
| C12645 | 2″′-N-Acetyl-6″′-deamino-6″′-hydroxyneomycin C | 657.6005 | 0.14 | 1.14E−02 | Down | 0.18 | −2.49 |
| C01750 | N-Acetylmuramate | 293.2793 | 30.52 | 1.67E−02 | Up | 2.26 | 1.18 |
| C05271 | Sedoheptulose 1,7-bisphosphate | 370.2805 | 15.32 | 2.46E−02 | Up | 2.18 | 1.12 |
| C05264 | beta-Alanyl-CoA | 837.7265 | 38.48 | 2.72E−02 | Down | 0.29 | −1.77 |
| C00234 | (S)-Hydroxydecanoyl-CoA | 937.6874 | 22.2 | 2.85E−02 | Up | 2.01 | 1.01 |
| C00798 | Pelargonidin 3-O-rutinoside 5-O-beta-D-glucoside | 740.7643 | 38.28 | 3.36E−02 | Down | 0.33 | −1.62 |
| C01222 | GDP-4-dehydro-6-deoxy-D-mannose | 587.162 | 5.11 | 3.57E−02 | Up | 2.07 | 1.05 |
| C04824 | trans-Hex-2-enoyl-CoA | 862.7897 | 0.3 | 4.17E−02 | Down | 0.18 | −2.45 |
| C02713 | Formyl-CoA | 794.7692 | 0.31 | 4.36E−02 | Up | 4.08 | 2.03 |
| C00447 | 2-Deoxy-D-ribose 1-phosphate | 214.0949 | 5.96 | 4.97E−02 | Down | 0.46 | −1.11 |
| C07547 | (24E)-3alpha,7alpha-Dihydroxy-5beta-cholest-24- | 1181.7054 | 38.13 | 6.03E−02 | Down | 0.50 | −1.01 |
| | enoyl-CoA | | | | | | |
| C00029 | FAD | 785.6484 | 0.39 | 8.52E−02 | Down | 0.38 | −1.41 |

^{†}Mass to charge ratio,
^{‡}Retention time,
^{§}Fold change with the threshold set at 1.0

All differentially regulated metabolites in vpp5 line as compared to WT were identified using KEGG database (Kyoto Encyclopedia of Genes and Genomes) (Tables 3, 4). The majority of identified rice metabolites can be linked to specific metabolic pathways using KEGG metabolic pathway enrichment analysis (FIGS. 5B, 5D, 5F, 5H). The 63 downregulated metabolites in 15 DAF grains of the vpp5-6 line consisted of primary metabolites such as cofactors (n=3), sugars and derivatives (n=6), nucleotides and nucleotide synthesis (n=7), and secondary metabolites such as metabolites related to alkaloid biosynthesis (n=6), folate biosynthesis (n=3), and porphyrin biosynthesis (n=4) among others. Of the 21 upregulated metabolites, 7 are related to primary metabolic processes that participate in the TCA cycle, nucleotide biosynthesis, L-aspartate, fatty acid biosynthesis, and a major cofactor in the anabolic process (NADP+). The rest are associated with mostly secondary metabolic processes or play important roles in regulating metabolic processes (FIG. 5b; Table 4).

TABLE 4
Differentially regulated metabolites in 15 DAF grains of vpp5-6 vs 10 DAF
grains of WT rice lines.

| KEGG | | | | | | | log2 |
|---|---|---|---|---|---|---|---|
| ID | Compound | m/z^{†} | RT^{‡} | p value | Change | FC^{§} | (FC) |
| C02134 | Allocryptopine | 369.404 | 15.37 | 2.29E−11 | Down | 0.12 | −3.02 |
| C00500 | Biliverdin | 582.528 | 17.79 | 2.53E−10 | Down | 0.18 | −2.45 |
| C01204 | Phytic acid | 659.453 | 18.26 | 3.14E−09 | Down | 0.15 | −2.75 |
| C01944 | Octanoyl-CoA | 892.801 | 22.51 | 2.35E−08 | Down | 0.37 | −1.45 |
| C05264 | S)-Hydroxydecanoyl-CoA | 936.835 | 20.48 | 2.43E−08 | Down | 0.25 | −2.02 |
| C16567 | Homotrypanothione | 737.658 | 19.62 | 3.91E−08 | Down | 0.13 | −2.99 |
| C04628 | Coenzyme B | 343.403 | 15.53 | 6.23E−08 | Down | 0.12 | −3.02 |
| C01138 | Streptomycin 6-phosphate | 661.537 | 18.29 | 1.58E−07 | Down | 0.06 | −4.14 |
| C00068 | Thiamin diphosphate | 425.372 | 16.28 | 1.62E−07 | Down | 0.04 | −4.78 |
| C00024 | Acetyl-CoA | 808.726 | 19.46 | 2.10E−07 | Down | 0.26 | −1.94 |
| C00075 | UTP | 484.46 | 17.22 | 3.33E−07 | Down | 0.12 | −3.10 |
| C00460 | UTP | 468.47 | 19.12 | 6.07E−07 | Down | 0.43 | −1.21 |
| C04824 | Lipid X | 711.749 | 19.72 | 8.64E−07 | Down | 0.21 | −2.22 |
| C05773 | Cobyrinate | 938.728 | 22.2 | 2.18E−06 | Down | 0.30 | −1.73 |
| C00332 | Acetoacetyl-CoA | 850.766 | 19.99 | 3.80E−06 | Down | 0.27 | −1.89 |
| C02106 | (S)−Scoulerine | 327.371 | 20.18 | 5.56E−06 | Down | 0.05 | −4.29 |
| C00100 | Propanoyl-CoA | 822.716 | 19.79 | 7.79E−06 | Down | 0.40 | −1.33 |
| C00689 | alpha, alpha′-Trehalose 6-phosphate | 422.424 | 20.59 | 8.00E−06 | Down | 0.05 | −4.30 |
| C08620 | Cyanidin 3-O-rutinoside | 595.538 | 17.91 | 1.85E−05 | Down | 0.16 | −2.63 |
| C03577 | 20-Hydroxyleukotriene E4 | 455.444 | 16.86 | 2.38E−05 | Down | 0.18 | −2.46 |
| C05952 | Leukotriene E4 | 439.581 | 19.93 | 3.13E−05 | Down | 0.08 | −3.62 |
| C11966 | Avermectin Alb monosaccharide | 728.592 | 19.45 | 3.53E−05 | Down | 0.30 | −1.76 |
| C00191 | D-Glucuronate | 194.09 | 6.13 | 3.64E−05 | Down | 0.21 | −2.23 |
| C00019 | S-Adenosyl-L-methionine | 398.393 | 16.08 | 4.15E−05 | Down | 0.10 | −3.29 |
| C00504 | Folate | 441.511 | 20.31 | 4.29E−05 | Down | 0.15 | −2.70 |
| C00061 | FMN | 456.478 | 17.2 | 4.37E−05 | Down | 0.19 | −2.36 |
| C00397 | Tobramycin | 467.509 | 19.8 | 7.13E−05 | Down | 0.16 | −2.68 |
| C00842 | dTDP-glucose | 564.348 | 17.08 | 9.67E−05 | Down | 0.50 | −1.00 |
| C05322 | 7-O-Acetylsalutaridinol | 371.374 | 15.92 | 1.13E−04 | Down | 0.50 | −1.01 |
| C05625 | Rutin | 610.553 | 18.59 | 1.18E−04 | Down | 0.13 | −2.99 |
| C00179 | Agmatine | 129.98 | 9.45 | 1.24E−04 | Down | 0.11 | −3.22 |
| C04494 | (Guanosine 3′-diphosphate 5′-triphosphate | 682.681 | 19.22 | 1.59E−04 | Down | 0.33 | −1.59 |
| C02166 | Leukotriene C4 | 625.603 | 18.7 | 1.67E−04 | Down | 0.30 | −1.76 |
| C00427 | Prostaglandin H2 | 352.31 | 15.66 | 2.69E−04 | Down | 0.15 | −2.74 |
| C00294 | Inosine | 268.201 | 15.57 | 2.75E−04 | Down | 0.12 | −3.00 |
| C04895 | 7,8-Dihydroneopterin 3′-triphosphate | 495.41 | 18.13 | 3.50E−04 | Down | 0.33 | 1.58 |
| C02074 | Raucaffricine | 512.514 | 18.69 | 3.81E−04 | Down | 0.12 | −3.03 |
| C22339 | Heme d1 | 709.638 | 19.2 | 3.95E−04 | Down | 0.20 | −2.35 |
| C00091 | Succinyl-CoA | 867.585 | 19.46 | 4.11E−04 | Down | 0.28 | −1.84 |
| C00131 | dATP | 491.298 | 18.55 | 7.07E−04 | Down | 0.56 | −0.85 |
| C00320 | Thiosulfate | 112.896 | 18.62 | 8.73E−04 | Down | 0.30 | −1.76 |
| C15681 | Mycinamicin III | 681.628 | 19.07 | 1.75E−03 | Down | 0.41 | −1.29 |
| C12032 | Clorobiocin | 695.714 | 19.66 | 2.12E−03 | Up | 2.99 | 1.58 |
| C00063 | CTP | 483.424 | 17.18 | 3.22E−03 | Down | 0.12 | −3.01 |
| C01304 | 2,5-Diamino-6-(5-phospho-D-ribosylamino) | 353.318 | 16.05 | 3.33E−03 | Down | 0.28 | −1.84 |
| | pyrimidin-4(3H)-one | | | | | | |
| C00083 | Malonyl-CoA | 853.415 | 5.91 | 3.54E−03 | Up | 1.91 | 0.93 |
| C05177 | Berbamunine | 596.587 | 18.13 | 3.68E−03 | Down | 0.25 | −1.98 |
| C00097 | L-Cysteine | 120.908 | 21.86 | 3.89E−03 | Down | 0.74 | −0.43 |
| C16290 | Delphinidin 3-O-3″,6″-O-dimalonylglucoside | 637.242 | 18.76 | 4.38E−03 | Down | 0.61 | −0.70 |
| C00315 | Spermidine | 145.054 | 6.24 | 5.78E−03 | Up | 2.56 | 1.36 |
| C01230 | all-trans-Hexaprenyl diphosphate | 586.37 | 30.08 | 5.97E−03 | Up | 1.67 | 0.74 |
| C01050 | UDP-N-acetylmuramate | 678.691 | 19.96 | 7.40E−03 | Up | 3.66 | 1.87 |
| C01595 | Linoleate | 280.35 | 35.09 | 9.79E−03 | Down | 0.42 | −1.26 |
| C00593 | Sulfoacetaldehyde | 123.973 | 6.04 | 1.02E−02 | Up | 3.94 | 1.98 |
| C01917 | Gentamicin A | 468.489 | 18.49 | 1.11E−02 | Down | 0.54 | −0.90 |
| C00513 | CDP-glycerol | 477.406 | 31.98 | 1.14E−02 | Up | 8.25 | 3.04 |
| C03329 | (S)-Canadine | 339.273 | 32.41 | 1.25E−02 | Down | 0.55 | 0.87 |
| C05923 | 2,5-Diaminopyrimidine nucleoside triphosphate | 513.274 | 33.6 | 1.30E−02 | Up | 1.42 | 0.50 |
| C02090 | Trypanothione | 723.376 | 31.59 | 1.38E−02 | Up | 2.45 | 1.29 |
| C03028 | Thiamin triphosphate | 505.354 | 15.91 | 1.47E−02 | Down | 0.40 | −1.32 |
| C00501 | CDP-glucose | 565.41 | 34.86 | 1.85E−02 | Up | 4.15 | 2.05 |
| C05273 | trans-Tetradec-2-enoyl-CoA | 975.574 | 32.75 | 1.95E−02 | Up | 1.88 | 0.91 |
| C04576 | Pentagalloylglucose | 939.772 | 21.42 | 2.12E−02 | Up | 3.55 | 1.83 |
| C00257 | D-Gluconic acid | 196.079 | 32.28 | 2.15E−02 | Down | 0.44 | −1.18 |
| C04536 | Magnesium protoporphyrin monomethyl ester | 598.582 | 18.55 | 2.30E−02 | Up | 1.80 | 0.85 |
| C01822 | Kanamycin A | 484.502 | 17.15 | 2.34E−02 | Down | 0.21 | −2.23 |
| C00006 | NADP+ | 743.622 | 38.27 | 2.41E−02 | Up | 1.69 | 0.75 |
| C00845 | 2-Furoyl-CoA | 861.371 | 21.42 | 2.42E−02 | Down | 0.64 | −0.64 |
| C00272 | Tetrahydrobiopterin | 241.062 | 5.49 | 2.66E−02 | Up | 1.74 | 0.80 |
| C00447 | Sedoheptulose 1,7-bisphosphate | 370.349 | 15.34 | 2.71E−02 | Down | 0.24 | −2.04 |
| C05414 | all-trans-Phytofluene | 542.415 | 38.01 | 2.71E−02 | Up | 1.72 | 0.78 |
| C00020 | AMP | 347.356 | 35.3 | 3.06E−02 | Down | 0.85 | −0.24 |
| C05774 | Cobinamide | 989.593 | 32.91 | 3.08E−02 | Down | 0.60 | −0.73 |
| C00188 | L-Threonine | 118.927 | 15.99 | 3.32E−02 | Down | 0.49 | −1.04 |
| C00354 | D-Fructose 1,6-bisphosphate | 340.327 | 32.28 | 3.61E−02 | Down | 0.79 | −0.34 |
| C00566 | (3S)-Citryl-CoA | 940.821 | 21.16 | 3.74E−02 | Up | 1.24 | 0.31 |
| C00029 | UDP-glucose | 566.43 | 36.95 | 4.15E−02 | Down | 0.33 | −1.58 |
| C04738 | UDP-3-O-(3-hydroxytetradecanoyl)-N- | 833.675 | 10.94 | 4.31E−02 | Down | 0.29 | −1.77 |
| | acetylglucosamine | | | | | | |
| C00049 | L-Aspartate | 133.009 | 5.99 | 4.32E−02 | Up | 1.67 | 0.74 |
| C00877 | Crotonoyl-CoA | 834.727 | 38.12 | 4.38E−02 | Down | 0.32 | −1.63 |
| C00387 | Guanosine | 283.389 | 38.19 | 4.60E−02 | Up | 2.66 | 1.41 |
| C00672 | 2-Deoxy-D-ribose 1-phosphate | 214.13 | 5.99 | 4.66E−02 | Up | 2.02 | 1.02 |
| C00234 | 10-Formyltetrahydrofolate | 473.424 | 35.6 | 4.72E−02 | Up | 1.28 | 0.36 |
| C04631 | UDP-N-acetyl-3-(1-carboxyvinyl)-D-glucosamine | 677.325 | 31.79 | 4.80E−02 | Down | 0.79 | −0.34 |

Notably, phytic acid, a major source of Pi in the seed, is 6.7-fold downregulated in vpp5-6 (Table 4), indicating possible effects on numerous enzymatic processes, including starch biosynthesis. Edwards et al. (2017) reported a major chalk QTL on low phytic acid gene in Kaybonnet mutant, KBNT1-1, a long grain tropical japonica rice, and suggested that low phytic acid could influence chalk formation. Interestingly, in our study, lower phytic acid was found in the 15 DAF grains of the translucent vpp5-6 line. This finding does not reflect on the phytic acid content of the mature grains. However, downregulation of phytic acid could point to the modulation of major metabolic pathways in the developing seeds of vpp5-6, resulting in lower chalkiness.

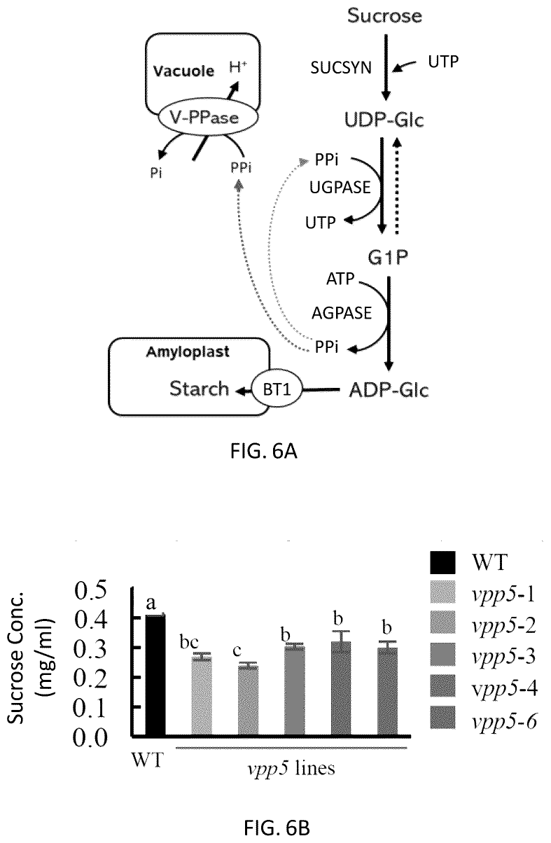

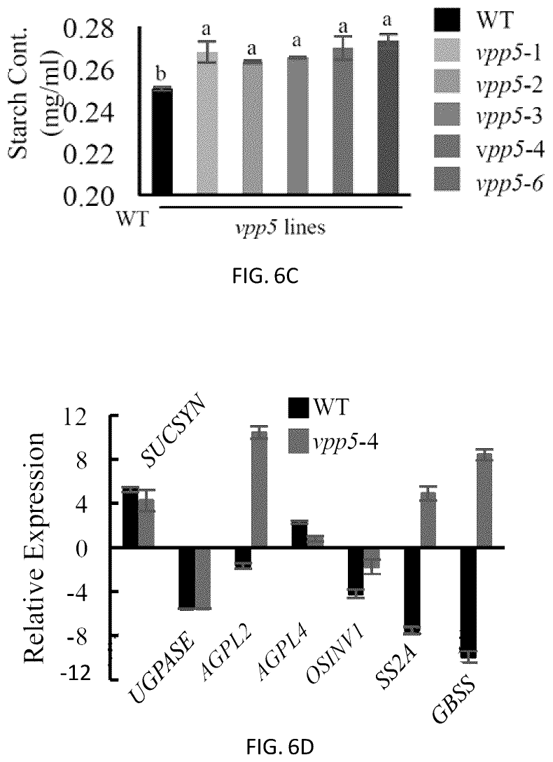

Next, UDP-Glucose, the primary metabolite in starch biosynthesis is 3.6-fold upregulated in 10 DAF grains but ~3-fold downregulated in the 15 DAF grains of vpp5-6 (Table 3, 4), indicating altered dynamics of starch biosynthesis in the vpp5. Starch biosynthesis starts with the transportation of sucrose from the leaf into endosperm, and its conversion to UDP-Glucose (UDP-Glc), leading to the production of ADP-glucose (ADP-Glc) that is transported into plastids, where it serves as the substrate for starch biosynthesis (FIG. 6A). Thus, downregulation of UDP-Glc in 15DAF grains could indicate lower sucrose availability. Interestingly, trehalose-6-phosphate (T6P) was highly downregulated (20-fold) in vpp5-6 (Table 4). It is widely accepted that T6P is a signaling molecule that plays a central role in carbohydrate metabolism (Paul et al., 2008; Ponnuet al., 2011), and the abundance of T6P is directly related to the amounts of UDP-Glc. T6P is, therefore, considered an indicator of sucrose availability in plants (Lunn et al., 2006). Corroborating with this, sucrose was significantly downregulated in vpp5 compared to the WT (FIG. 6B).

Overall, the suppression of V-PPase activity in the developing grains appears to alter the rate of major metabolic processes. In this context, it is important to note that spermidine was 2.5-fold upregulated in vpp5-6 grains (Table 4). Spermidine accelerates endosperm cell division and grain filling in rice by modulating the activities of key enzymes involved in starch and amino acid biosynthesis, resulting in higher starch and amino acids contents in the mature rice grains (Xu et al., 2021; Yang et al., 2008). This led us to determine the effect of vpp5 mutation on total starch content and the expression of starch biosynthesis genes. The starch content in the mature vpp5grains was significantly higher than that of the WT (p<0.01), which was correlated with the upregulation of key starch biosynthesis genes (FIG. 6C-D). Specifically, real time quantitative PCR using total RNA of 10 DAF grains showed that the major starch biosynthesis genes consisting of cytosolic ADP-glucose pyrophosphorylase (AGPase) subunit gene AGPL2, Starch Synthase IIA (SS2A) and Granule Bound Starch Synthase I (GBSSI) are abundantly upregulated in vpp5 line. The expression of Sucrose Synthase 3 (SUCSYN) and UDP-glucose pyrophosphorylase (UGPASE) was unaltered, and that of Cell Wall Invertase (OCIN1) and plastidial AGPase subunit gene AGPL4 was downregulated in vpp5 (FIG. 6D). These results corroborate with the findings of Fu et al. (2019), who reported improved heat tolerance in rice by exogenous application of spermidine, leading to improved seed quality (lower chalk), starch content, and enhanced expression of starch biosynthesis genes.

Grain chalkiness reduces the yield of the milled rice by increasing the proportion of broken rice. Numerous chalk QTLs have been described, many of which are located close to the genes controlling grain size or grain width (Dwiningsih et al., 2021; Li et al., 2014; Sreenivasulu et al., 2015). The high number of QTLs and their association with the yield traits complicates the breeding for low chalk rice.

Chalk5 is a major QTL identified in the indica rice population that encodes a vacuolar H+-translocating pyrophosphatase (V-PPase) (Li et al., 2014). Overexpression of Chalk5 due to the presence of two cis-elements (RY/G and CACT) located in its promoter is the molecular basis of the chalkiness. Consistent with their findings, we located RY/G and CACT in the putative Chalk5 promoter sequences of two indica varieties, ZHE 733 and Teqing, that are well-known chalky varieties. These cis-elements are absent in the japonica Chalk5 allele, referred to as VPP5, in this study. We sequenced 960 bp of the VPP5 promoter (upstream of start codon) from 19 japonica rice including Nipponbare and 18 elite cultivars derived from tropical japonica subspecies and found no sequence polymorphism (Data not shown). However, variation in the grain chalkiness has been reported in these cultivars by others (Esguerra et al., 2020; P. A. Counce, pers. comm.) and on a subset of these varieties in our study. The sensitivity to HNT is also highly variable in these cultivars, some of which (e.g., LaGrue) turn very chalky under HNT conditions. Therefore, strategies to control chalkiness is needed to improve the production of elite japonica cultivars under warmer climate.

As described above, polymorphism in VPP5 sequence has not been linked with the natural variation in the chalkiness of japonica rice. However, we found differential pattern of VPP5 expression in the grains at 10 DAF (FIG. 2). Li et al (2014) demonstrated increased grain chalkiness in a japonica rice (Zhonghua 11) by overexpressing VPP5 using the transgenic approach. Therefore, mechanism of chalkiness controlled by VPP5 is not unique to the long grain indica rice but it's expression in the small grain japonica rice could also account for grain chalkiness. The VPP5 expression pattern in Nipponbare was similar to that of ZHE 733, the chalky indica variety. Nipponbare grains developed 12.93% chalk that increased to 18.36% under HNT (Table 1). To evaluate whether VPP5 expression contributes to chalkiness in Nipponbare, we carried out promoter mutagenesis using CRISPR/Cas9. The recovered vpp5 mutant lines all harbored identical mutation that disrupted a GATA element in the promoter, resulting in the downregulation of VPP5 (FIG. 3). Most interestingly, the grain of vpp5 lines consistently showed significantly lower chalk in the control and HNT condition (Table 2). Multiple GATA transcription factor (TF) regulated genes are found in rice genome, all of which bind the consensus GATC motifs in rice (Reyes et al., 2004; Franco-Zorilla et al., 2014). The AGATCT motif is recognized by 9 GATA TFs in rice. Based on this, we propose that AGATCT motif is responsible for VPP5 expression in rice. However, rice GATA TFs are not well-characterized except OsGATA12 that is associated with increased growth and yield and regulates senescence related genes (Lu et al., 2017).

The mechanism of VPP5-mediated grain chalkiness is not clear. Li et al. (2014) studied hyperactivity of VPP5 and suggested that increase in the H+ concentration could lead to water loss and disturbance in the pH homeostasis of the endomembrane systems, resulting in the increase in the vesicle like structures and decrease in protein bodies number and size, manifesting loose arrangements of the starch granules. Li et al. (2014) also found that major genes involved in starch metabolism as well as two genes encoding storage proteins were significantly upregulated by overexpression of Chalk5 (VPP5). In our study, suppression of VPP5 was associated with the upregulation of starch biosynthesis genes; however, this upregulation occurred in a coordinated manner that presumably support efficient starch biosynthesis and packing in the endosperm.

Coordinated upregulation of AGPase, GBSS1, and SS2A is associated with the formation of polyhedral shaped granules that pack tightly despite HNT treatment (Gann et al., 2021). Downregulation of starch biosynthesis genes by heat, on the other hand, is known to disturb starch biosynthesis and produce chalky grain (Dhatt et al., 2019; Fu et al., 2019; Phan et al., 2013). Since many elite cultivars of rice succumb to heat-induced chalkiness (Esguerra et al., 2021; Lanning et al., 2011; Lyman et al., 2013; Morita et al., 2016), heat tolerance is an important trait to breed. In this context, upregulation of spermidine in vpp5 lines is notable. Spermidine positively regulates heat tolerance. Accordingly, heat-tolerant cultivars contain higher spermidine in the grains during early filling stages (Cao et al., 2016; Fu et al. 2019; Wang et al., 2012). Finally, since starch biosynthesis is fueled by PPi, which is hydrolyzed by V-PPase (FIG. 6A), higher activity of V-PPase could arguably deplete PPi and disturb starch biosynthesis process, leading to chalky grains.

Overall, suppression of VPP5 correlated with adjusted rate of metabolic processes including starch biosynthesis that arguably favors the production of large granules that pack tightly and prevent air spaces, eventually leading to the formation of low chalk, translucent grains. In conclusion, VPP5 plays a major role in the grain chalkiness in rice and its suppression through targeted mutagenesis of the promoter elements could generate favorable alleles for breeding HNT tolerant rice.

### EXPERIMENTAL PROCEDURES

Plant Materials. Seeds of five genotypes, Nipponbare, LaGrue, Taggart, ZHE 733 and Diamond, were obtained from Rice Research and Extension Center, Stuttgart, AR. All plant growth and treatments were done according to Esguerra et al. (2019). Six seeds of each genotype were germinated and grown in the greenhouse. Three replicates for each genotype were distributed to the two growth chambers upon reaching reproductive stage, R0-R2, according to Moldenhaueret al. (2018). The control chamber was set at 30° C. during daytime (6 AM to 8 PM) and 22° C. during nighttime (8 PM to 6 AM). The HNT chamber followed the same setting except for a higher nighttime temperature of 28° C. Relative humidity and lighting conditions were uniform for the two set-ups. Similarly, tissue culture derived wild-type (WT) Nipponbare (representing three independent lines) and vpp5 lines were grown in the greenhouse or growth chambers.

Grain Characterization. Grains from the different planting periods were characterized in terms of chalkiness, weight per 1000 grains, and grain filling rate. For chalkiness, 150 grains were collected 40 days after flowering (DAF), dried for two weeks at room temperature and analyzed for chalkiness using WinSEEDLE™. Chalkiness is expressed as the percent average area of the white opaque portion (chalk) in the grain relative to the total area of the grain. In determining the weight per 1000 grains, 500 grains were used from each of the three biological replicates and the average weight were multiplied by two. Grain filling rate was measured using the first three panicles from each plant. It is expressed as the percentage of filled grains from the total number of florets in the panicle.

Gene expression analysis. Total RNA from the caryopses from different genotypes and treatments was isolated using Trizol (Invitrogen Inc.) and quantified using Nano-drop 2000 (Thermo-FisherInc). Two micrograms of total RNA were treated with RQ1-RNAse free DNase (ThermofisherInc.), and one microgram of the DNase-treated RNA was used for cDNA synthesis using PrimeScript RT reagent kit (Takara Bio, CA, USA). The expression analysis was performed using TB green Premix Ex Taq II (Takara Bio, CA, USA) on Bio-Rad CFX 96 C1000 with following conditions: 95° C. for 30 sec and 40 cycles of 95° C. for 5 sec+60° C. for 30 sec. The product specificity was verified by the melt curve analysis. Rice 7Ubiquitin fused protein gene was used as the reference gene. Primer sequences used in this study are given in Table 5.

TABLE 5
Primers used in this study (5′ to 3′).

| Use | Gene | | Sequence | |
|---|---|---|---|---|
| qPCR | VPP5 | Forward: | CACCTGTTCATCTGCGTCTC | (SEQ ID NO: 8) |
| | | Reverse: | CGTAAGCATTGCTCGTGAAGTA | (SEQ ID NO: 9) |
| | SUCSYN | Forward: | GGAGTATGTGAGGGTCAATGTG | (SEQ ID NO: 10) |
| | | Reverse: | GTTGTTGTTGGTGCCTTCTTC | (SEQ ID NO: 11) |
| | UGPASE | Forward: | CCCGAGATATCCCTGAGATTAGA | (SEQ ID NO: 12) |
| | | Reverse: | CCTTACCTTTCTTGACCTTGCTA | (SEQ ID NO: 13) |
| | AGPL2 | Forward: | ACTGAGGAAGAGGTGCTTTG | (SEQ ID NO: 14) |
| | | Reverse: | GAGGATTGTGTCCGAAGATGAG | (SEQ ID NO: 15) |
| | AGPL4 | Forward: | GCAAGAAGCAGAGAGACCATTA | (SEQ ID NO: 16) |
| | | Reverse : | AACTGTACCATCTGGAATCACC | (SEQ ID NO: 17) |
| | OSINV1 | Forward: | GACCTGCACGAGCACAC | (SEQ ID NO: 18) |
| | | Reverse: | GTCAAGTCGGTGCACATAAGA | (SEQ ID NO: 19) |
| | SS2A | Forward: | GAGACGTACCGCAAGTACAA | (SEQ ID NO: 20) |
| | | Reverse: | GACAAGGACCTCCTCGTAGA | (SEQ ID NO: 21) |
| | GBSS1 | Forward: | GGTACTGGAAAGAAGAAGTTCG | (SEQ ID NO: 22) |
| | | Reverse: | TCCGGCCATGATGAGATGAGCAA | (SEQ ID NO: 23) |
| | 7UBIO | Forward: | TGGTCAGTAATCAGCCAGTTTG | (SEQ ID NO: 24) |
| | | Reverse: | CAAATACTTGACGAACAGAGGC | (SEQ ID NO: 25) |
| Sequencing | VPP5 | Forward: | AACCGAACGCGTCCTAAGTT | (SEQ ID NO: 26) |
| | promoter | Reverse: | GAGGAGAAGACGAGCGAGTG | (SEQ ID NO: 27) |

Granule Structure. Grains were harvested at 40 DAF and dried for two weeks at room temperature prior to viewing. A cross section of each grain were used to capture micrographs of the granules in the translucent and chalky portion using a Philips/Fei XL-30 environmental scanning electron microscope with the settings, Acc V. of 10 kV, 2000× magnification, 3.0 spot and 10 μm bar. Micrographs were adjusted to the brightness of 20, color balance of R (0), G (0), and B (−20), and gamma correction of 1.00.

Sucrose and Starch Content. Four grains per line were collected at 10 DAF in 1.5 ml tubes, immediately frozen in liquid nitrogen, and powdered with a plastic pestle for sucrose analysis. Sucrose was extracted from 25 mg of each sample in three replicates by adding 200 ul of 80% ethanol in each sample, and incubation of 10 days at 37° C. The samples were then centrifuged at 5,000 g for 10 minutes and the supernatant were transferred to new tubes. Sucrose content was determined using resorcinol (0.05% resorcinol in 3N HCl) method and estimation was based on absorbance at 420 nm using Cytation™3 and a standard curve. Starch was extracted from mature grains (4 grains per line) in three replications following the starch pre-treatment and determination using manufacturer's protocol (Megazyme K-AMYL kit).

Data Analysis. Grain characteristics, sucrose and starch content were laid out in a completely randomized design with three biological replications for each genotype or line. Data in percentages and proportions were transformed using arcsine and were subjected to one-way ANOVA. Means were compared for significant differences using Tukey's multiple comparison test. Statistical analyses were carried out in SAS statistical software (version 9.4, SAS Institute Inc.).

Total Plant Metabolome by LC-MS. Rice grains (10 DAF and 15 DAF) were frozen in liquid nitrogen. Hulls were removed from the grains, and four equal-sized grains from each sample were placed in 1.5 mL microcentrifuge tubes containing 80% LC-MS-grade methanol and 1 mm cubic silica-zirconia beads. Tubes were sealed with parafilm and loaded into a Fisherbrand Bead mill 24bead beater, allowed to homogenize for five cycles at 2.9 M/s for 60 seconds each with a pause dwell time of 15 seconds between each cycle, until the solution became milky and approached opaque. Six biological replicates (consisting of four grains each) were processed. Samples were then transferred with a syringe into fresh 1.5 mL tubes, vortexed for one minute, sonicated for fifteen minutes, and centrifuged at 10,000 rpm for 5 minutes. The resulting supernatant was then filtered using 0.2 μm TARGET2 syringe filters. The tubes were transferred to a vacufuge to dry overnight at 45° C., and then reconstituted in 80% LC-MS grade methanol. Samples were filtered again using syringe filters and transferred directly into glass autosampler vials. LC-MS processing of samples was conducted as previously described (Rezaei Cherati et al. 2022). Raw files were loaded into MZmine3 software (http://mzmine.github.io/) and processed as was described by Pluskal et al. (2010). MZmine3 provided initial compound annotation after alignment of peaks and peak gap-filling using the KEGG online database (Ogata et al. 1999). The mass peak lists were uploaded to Metaboanalyst (www.metaboanalyst.ca/) for further statistical analysis and pathway enrichment (Xia et al. 2015).

ACCESSION NUMBERS: VPP5 (Os05g0156900), SUCSYN3 (Os03g0401300), OSINVI (Os09g0255000), UGPASE (Os05g0468600), AGPL2 (Os01g0633100), AGPL4 (Os07g0243200), GBSSI (Os06g0133000), SS2A (Os06g0229800)

### Example II: Creation of Oryza sativa Kitaake Line with Reduced VPP5 Expression and Reduced Chalkiness

The effect of targeting VPP5 in japonica rice variety Kitaake was also investigated to demonstrate that a similar result would be found in other rice lines. Kitaake bears 100% sequence homology with Nipponbare-VPP5 gene in the promoter region. However, Kitaake-VPP5 contains ~1 kb insertion in its 1st intron, so it might be regulated differently than Nipponbare-VPP5.

### Characterization of Targeted Lines

Transformation of Kitaake was done with the CRISPR/Cas9 vector used for Nipponbare (described above). Transformation of rice with a CRISPR/Cas9 vector containing two sgRNAs, generated six primary transgenic lines, only one of which carried targeted mutation in the VPP5 gene. This line, called KT-6, contained the following homozygous mutation: deletion between the two target sites, with insertion of a small sequence. The sequence of the resulting mutation was 5′-CCAaagaagagtagcctttccaaG-3′ (SEQ ID NO: 28), in which the uppercase letters are the far ends of the target sites and small case letters are insertions. The Cas9 (−) progeny of this were selected and compared against a tissue culture-derived Kitaake line, KW-8.

### VPP5 Expression is Downregulated in KT-6

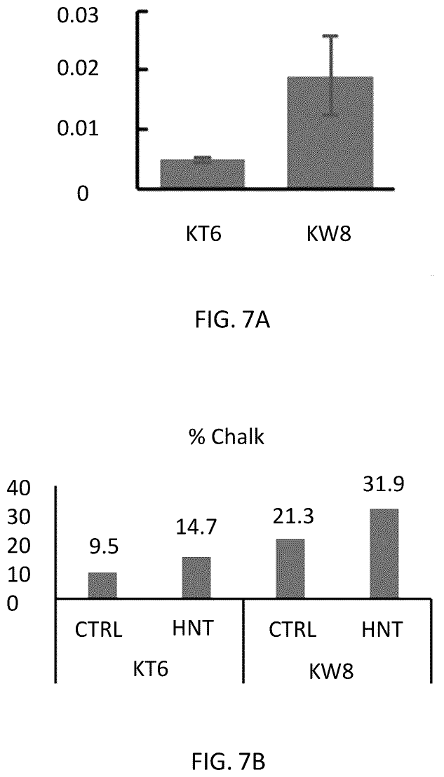

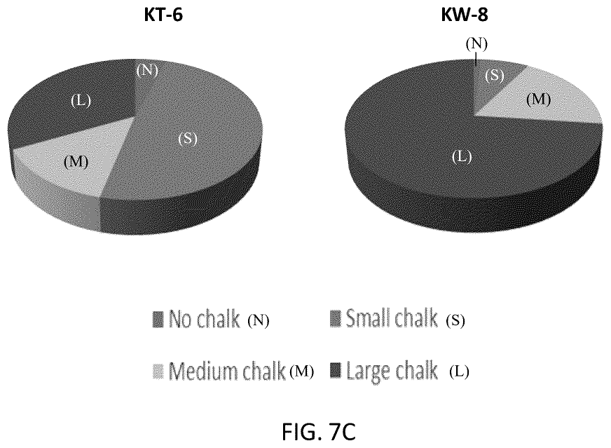

The expression of VPP5 in developing caryopses at 10 days after flowering (DAF) was analyzed by qRT-PCR on total RNA isolated for 10DAF caryopses. Three replicates per genotype were performed, and the expression was normalized against the rice 7Ubi gene using the delta CT method. The results indicated that expression of VPP5 was downregulated about 3.8-fold in the KT-6 mutant line as compared to the wild-type line, KW-8 (FIG. 7A).

### Grain Analysis of KT-6 and KW-8

T2 plants of KT-6 and KW-8 were grown in a greenhouse until booting stage, and then transferred to either a control chamber (30° C. day and 21° C. night) or an HNT chamber (30° C. day and 28° C. night) with a 14 hour daytime starting at 6 am and ending at 8 μm. Six plants of each genotype were grown in the greenhouse and when the first stem on each plant reached the R2 stage (flag leaf collar formation), the stem was tagged, and the plants were divided equally in the control and HNT chambers (3 plants each genotype/treatment). The mature seeds (40 DAF) from the plants were harvested and air-dried for 2 weeks at room temperature (~12% moisture content). The dried seeds were then subjected to grain chalk analysis using a WinSEEDLE™ Analysis System. At least 108 seeds were analyzed for each genotype/treatment. Higher chalk content was observed in KW-8 under both growing conditions (FIG. 7B). Notably, the chalk content in KT-6 was about 45% and 46% of the chalk content in KW-8 under control and HNT conditions, respectively.

Next, 108 grains from each genotype grown under HNT conditions were analyzed for chalk size. Chalk sizes were classified relative to the area of the grain as none (no chalk), small (<10%), medium (11%-20%), and large (>20%). The results showed that KT-6 was less susceptible to chalkiness under HNT conditions: KT-6 had a higher number of grains with no chalk or small chalk, while KW-8 had higher number of grains with large chalk (FIG. 7C).

### Yield

Finally, the effect of the VPP5 mutation on grain yield was determined by measuring the number of grains collected for KT-6 and KW-6 in each planting/treatment group, along with the weight of 1000 grains (Table 6). The analysis used 3 plants per genotype/treatment. The results did not demonstrate an apparent effect of treatment condition (Control vs. HNT) on either genotype. However, KT-6 produced more seeds than KW-8 under both treatment conditions. Seed weight was similar for both genotypes.

TABLE 6
KT-6 and KW-8 yield analysis

| | |
|---|---|
| Number of Grains | Weight (1000 grains) |

| Genotype | Control | HNT | Control | HNT |
|---|---|---|---|---|
| KW-8 | 108 | 134 | 18.5 | 20.5 |
| KT-6 | 155 | 149 | 19.5 | 19.5 |

### CONCLUSION

The newly generated VPP5 mutant line, KT-6, demonstrates that suppression of VPP5 in japonica rice variety Kitaake results in improved grain chalkiness in grains ripened under control or HNT conditions.

### Sequences

### Nipponbare VPP5 (Os05g0156900) Gene Sequence (SEQ ID NO: 29): 6195 bp

Chromosome 5:3319645 to 3313450

~1.8 kb upstream sequence included that contains the promoter and numerous cis-elements (none of which are characterized)
1) The bold sequence is targeted by CRISPR/Cas9
2) CCA (bold and underlined) is PAM sequence of the target site.
3) GATA motif is AGATCT.
4) Bold and larger font (ATC) is deleted in the gene edited lines
5) The TATA box is italicized
6) The transcriptional start is italicized and enlarged
7) The ATG translation start site is also italicized, underlined, and larger font
8) The exons are underlined.

AAAGCATGGCAACACGAAAATATAACCATGACACCATCACAAGCTCACAAAGATGGAAAT
GTTGTTGTATTTATCATTGGCATATTGCTCTAGGCCCACATATCATTCTCGACATGCAAA
ATGTTACTTTCCGGTTCATTACAAATTAATCTTTATTTCTCTACGGTTAAATTTCAGTTC
AAACATTTAAAGTGATAATTACTTGTCGTTCAAACCATCAAAATGTTGAAAGCGAGAAAG
GTTCATTACCAAAGAAATGTIGTCTTGAGGCCAAACGGGGCCTAAGGGCACGITTGGATC
CTGGGCAAGGCTCATCCTAACCTCGCTTAGTAAAGCTACACGTTTGGAACTGGTAAAATA
ATCTGGCTTTTGGTTTTGGGGACGAGCTAGCTTTGCAGGGCAAGAAAATTCTTGCTAGCC
AGTGAGAACCGATTTAGCTCTCCTTCGACGGCAAACTCCGTTCGCGTGAAAATATTGGAA
CAAGGGCGATGCAGACTTCTATGAAATTGGCGCCGTAAAATTAGCGATGTTTTTCAATGA
CTCACCCAATCTTGTCGGCCTCTTCTTCCAGATCTTGTTGTGAACTCTCGCTCGCTCAAA
TTTTGTTGTAAATTGCCCTCTCCACCAACCCATAAATACAACGAACAATCAGTCTGGATC
TAAAAGGAAAAATTGATTTATGGAAGAACTCAGCAACAAGAGGAACTCATCTGTAGCAGC
TAGCCATTAATCCTTGCCACAAGAAGCAAAACCATCCAAACAGAGGAGCTACAACAAGGC
TAGCAAGTTGGCAAATAGATGGTTTAGTGCTGCTAGGCTAGCTGGGCGAGCGAACCGAAC
GCGTCCTAAGITAATTTGTCCTATTATATCTAACCTCGACACTTGTACTGCACAAGACAC
TAGTGCGCTCCCTTGAACAGCTTTTTTTATGGCTTTTTCACTAGAAGCTGTAACAACTTT
GGTTTCTTTTGGCCATCTTTCCTCCAATTAGAACAGGATTGCATACATCTTAACAGCAAA
GAGAGATGCAAACTATTAAAGTTTATCATCACAGGAAAACTTATGACATTTACAACTTTG
AAATTGAAATATAGCAAGATAAATCAAGTAGCCTTTCCAAGTTTCTTTTGACCAAAGAAG
AGAAAAGTGCCAAAGATCTACATGACCCAGAGTTGTTTTATTCAAAAACCACCATTCGAA
ATGAAGGTAGCTAGATGCATGATGAGGGAAATTTTGTTGCATATGGGTGAACCCCAAAGC
TTACCTAATGGCATTGTCCGTCAGCATTTCCAGTGCGTCCAAACCTCAATGAGACGCACC
GTAGATAACTTATGCTCATGCGTCAGATACACCTAATTTGAACCGACGGTAAAGGATTCT
AGACGGTGCCGTTTGTAGCGGCTTTTTCGCTACTTTTCTTGGTGAGATAGAAGACAGTGA
CAAAATAAGTTGCAAAAACAACAAAAATGTTTATACACATGTACAGCATGAGCTCTACGG
TGTCCCTGGAGAGCACAAGCTGTCACCAAGCCATAACAAACGGTCGAACTGATCTTGAAC
ACTGGATCCAATGCATGGTTTGTTCAGTGCTCAAAGAGAACACCAGCAAACTTTCCATTT
AGACCCTCACAAGCCACAAATATGCGGAAAAAAATGTCATATTCACCCTCCAAAAAGCT
TATACAAGGACTTGITTGTAATTATCTTAAAATACAGGTGGGTAATTGCAAAGTGAGACA
TCTTCCCTTCTATAAAGAGCAAGCTTAGCTTGGTGATCACCACCACTCCACATCTCT
TCTCCATCAAGAATCAATCACTCGCTCGTCTTCTCCTCCATTGGAGCTCTCGATCGAGCT

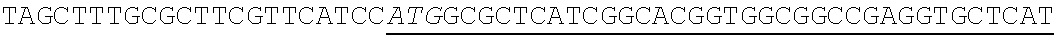

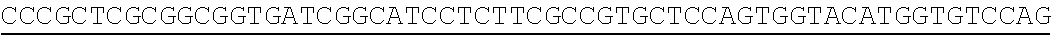

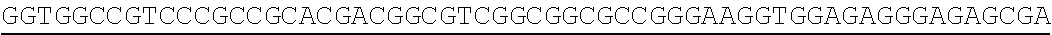

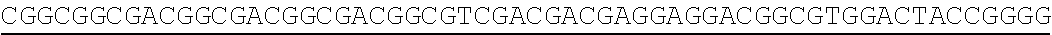

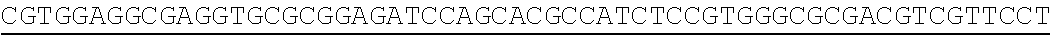

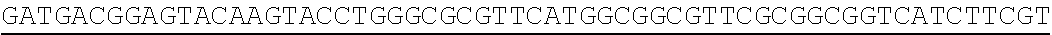

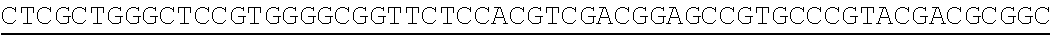

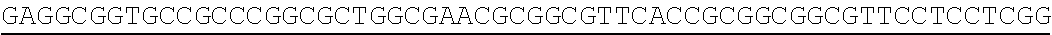

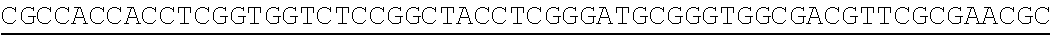

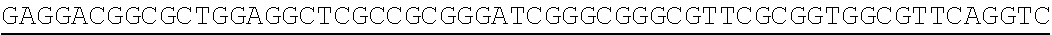

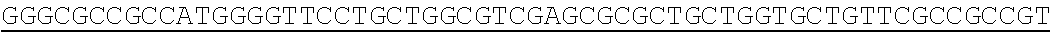

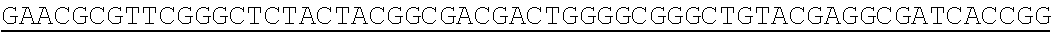

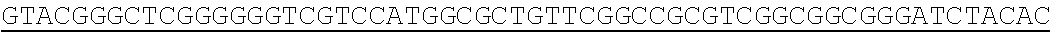

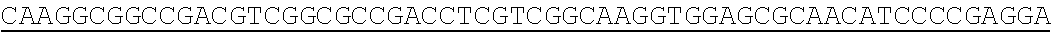

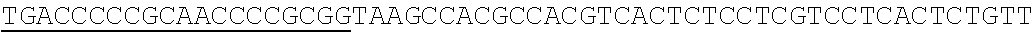

AAAGATTAAGTTAACACACATAAAAATTAAGTTAACGCACATGAAACGAGAAAGCT
ATTAGCATATAATTAACT
CCCATCGTTCCCTTCCTTCAAAGAGAAGTGATACATCATATATGGTTTTGTACGTGCGTG

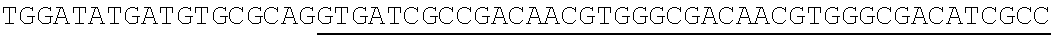

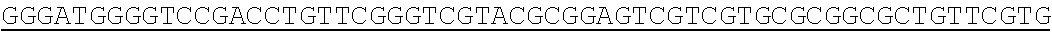

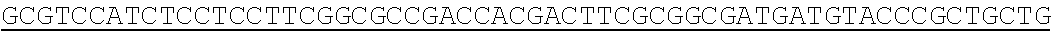

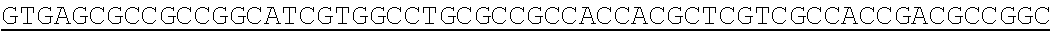

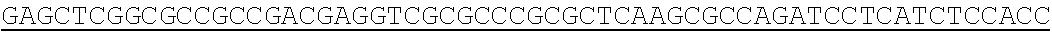

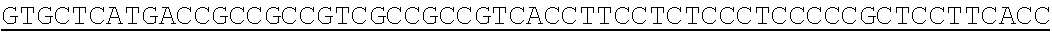

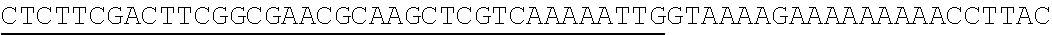

ATAGTGTGTAATTTATTTTTTTTTGGTTTTTTTAAAAAATTATAATTACACGGCAGACA
TACGCACACAAACAAGACCACACTCACATCCCTGTATACAATACATTTTTTTT
AAAAGTTTTACAATTACACGGGAGACACACGTGCACAGATAATACCACACTCACATCCCT
GTCTATGTGCATTCAACAACACATTCTTGATTTCGGAGAAATCATGTTAAAAACCTAGTT
ACAGTGCGTAATATTTATAAGCACCAAGATTTGAATCTTGTTGGGTGGAGCCATGCACCT
ATGACTCTAGCAGTTGTACTTGCACCAAATTTGATTAAATTATGGAATAATAATAGITTT

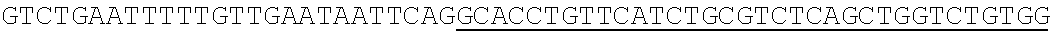

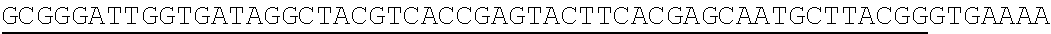

CGAATTCTACCCACCCAAATTCGCGATGATTCTACATTTTGTAAAATTTCAAATTTTGAT

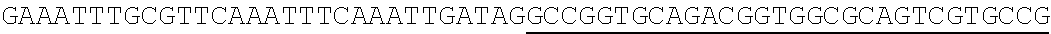

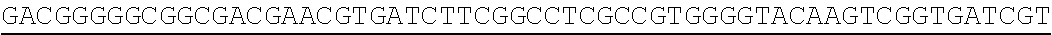

GCCGATCTTCGCCATCGCCGGCGCCATCTACGCCAGCTTCCGGCTCGCCGCCATGTACGG

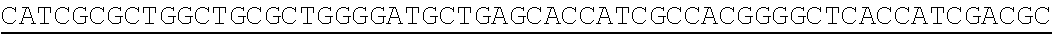

CTATGGCCCCATCAGCGACAACGCCGGCGGCATCGCGGAGATGGCCGGCATGCCGCGGCG

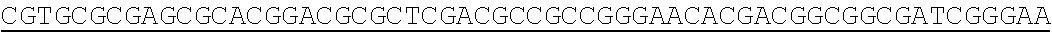

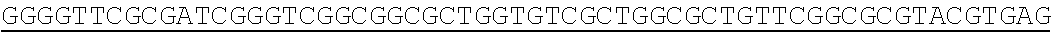

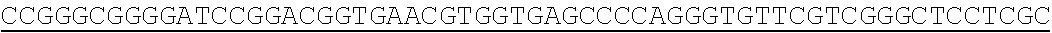

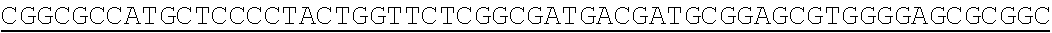

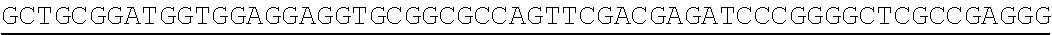

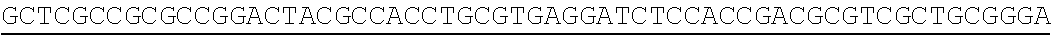

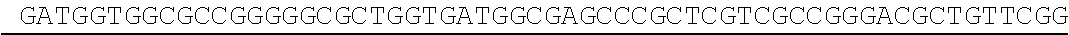

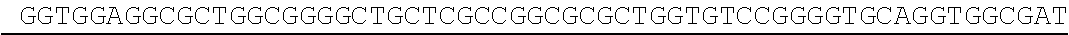

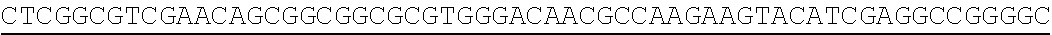

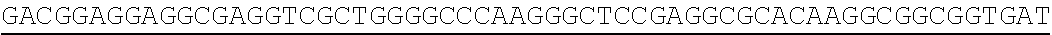

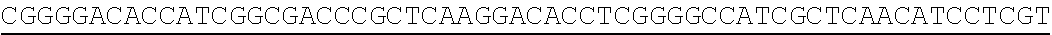

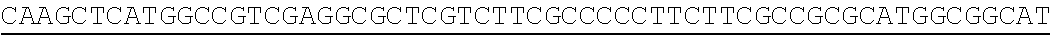

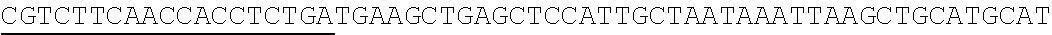

GCTCTCGCCATGATCGATCAGCTGCACCTGCATGCACGCATGGATAAACAAGAAGAAGAC
GATGAGCATTAGGGTTCCTTAACCAGTTGAAACCGATAAGAGTAATAATTGTAGCTCG
GGATAATGCAGTGTCAGATGTAATCCAAAGTGTCTTGTTGCTTGGGATATGTAATGC
GGTTTGAGACTT

Kitaake VPP5 (OsKitaake05g043600) gene sequence
(SEQ ID NO: 30): 6195 bp

```
  1. Insertion sequence in 1st intron in bold;
  2. Promoter target sites in italics;
  3. Exons underlined
AAAGCATGGCAACACGAAAATATAACCATGACACCATCACAAGCTCACAAAGATGG
AAATGTTGTTGTATTTATCATTGGCATATTGCTCTAGGCCCACATATCATTCTCGACA
TGCAAAATGTTACTTTCCGGTTCATTACAAATTAATCTTTATTTCTCTACGGTTAAAT
TTCAGTTCAAACATTTAAAGTGATAATTACTTGTCGTTCAAACCATCAAAATGTTGA
AAGCGAGAAAGGTTCATTACCAAAGAAATGTTGTCTTGAGGCCAAACGGGGCCTAA
GGGCACGTTTGGATCCTGGGCAAGGCTCATCCTAACCTCGCTTAGTAAAGCTACACG
TTTGGAACTGGTAAAATAATCTGGCTTTTGGTTTTGGGGACGAGCTAGCTTTGCAGG
GCAAGAAAATTCTTGCTAGCCAGTGAGAACCGATTTAGCTCTCCTTCGACGGCAAAC
TCCGTTCGCGTGAAAATATTGGAACAAGGGCGATGCAAACTTCTATGAAATTGGCGC
CGTAAAATTAGCGATGTTTTTCAATGACTCACCCAATCTTGTCGGCCTCTTCTTCCAG
ATCTTGTTGTGAACTCTCGCTCGCTCAAATTTTGTTGTAAATTGCCCTCTCCACCAAC
CCATAAATACAACGAACAATCAGTCTGGATCTAAAAGGAAAAATTGATTTATGGAA
GAACTCAGCAACAAGAGGAACTCATCTGTAGCAGCTAGCCATTAATCCTTGCCACA
AGAAGCAAAACCATCCAAACAGAGGAGCTACAACAAGGCTAGCAAGTTGGCAAAT
AGATGGTTTAGTGCTGCTAGGCTAGCTGGGCGAGCGAACCGAACGCGTCCTAAGTT
AATTTGTCCTATTATATCTAACCTCGACACTTGTACTGCACAAGACACTAGTGCGCTC
CCTTGAACAGCTTTTTTTATGGCTTTTTCACTAGAAGCTGTAACAACTTTGGTTTCTTT
TGGCCATCTTTCCTCCAATTAGAACAGGATTGCATACATCTTAACAGCAAAGAGAGA
TGCAAACTATTAAAGTTTATCATCACAGGAAAACTTATGACATTTACAACTTTGAAA
TTGAAATATAGCAAGATAAATCAAGTAGCCTTTCCAAGTTTCTTTTGACCAAAGAAG
AGAAAAGTGCCAAAGATCTACATGACCCAGAGTTGTTTTATTCAAAAACCACCATTCG
AAATGAAGGTAGCTAGATGCATGATGAGGGAAATTTTGTTGCATATGGGTGAACCC
CAAAGCTTACCTAATGGCATTGTCCGTCAGCATTTCCAGTGCGTCCAAACCTCAATG
AGACGCACCGTAGATAACTTATGCTCATGCGTCAGATACACCTAATTTGAACCGACG
GTAAAGGATTCTAGACGGTGCCGTTTGTAGCGGCTTTTTCGCTACTTTTCTTGGTGAGA
TAGAAGACAGTGACAAAATAAGTTGCAAAAACAACAAAAATGTTTATACACATGTA
CAGCATGAGCTCTACGGTGTCCCTGGAGAGCACAAGCTGTCACCAAGCCATAACAA
ACGGTCGAACTGATCTTGAACACTGGATCCAATGCATGGTTTGTTCAGTGCTCAAAG
AGAACACCAGCAAACTTTCCATTTAGACCCTCACAAGCCACAAATATGCGGAAAAA
AATGTCATATTCACCCTCCAAAAAGCTTATACAAGGACTTGTTTGTAATTATCTTAA
AATACAGGTGGGTAATTGCAAAGTGAGACATCTTCCCTTCTATAAAGAGCAAGCTTA
GCTTGGTGATCACCACCACTCCACATCTCTTCTCCATCAAGAATCAATCACTCGCTC
GTCTTCTCCTCCATTGGAGCTCTCGATCGAGCTTAGCTTTGCGCTTCGTTCATCCATG
GCGCTCATCGGCACGGTGGCGGCCGAGGTGCTCATCCCGCTCGCGGCGGTGATCGG
CATCCTCTTCGCCGTGCTCCAGTGGTACATGGTGTCCAGGGTGGCCGTCCCGCCGCA
CGACGGCGTCGGCGGCGCCGGGAAGGTGGAGAGGGAGAGCGACGGCGGCGACGGC
GACGGCGACGGCGTCGACGACGAGGAGGACGGCGTGGACTACCGGGGCGTGGAGG
CGAGGTGCGCGGAGATCCAGCACGCCATCTCCGTGGGCGCGACGTCGTTCCTGATG
ACGGAGTACAAGTACCTGGGCGCGTTCATGGCGGCGTTCGCGGCGGTCATCTTCGTC
TCGCTGGGCTCCGTGGGGCGGTTCTCCACGTCGACGGAGCCGTGCCCGTACGACGCG
GCGAGGCGGTGCCGCCCGGCGCTGGCGAACGCGGCGTTCACCGCGGCGGCGTTCCT
CCTCGGCGCCACCACCTCGGTGGTCTCCGGCTACCTCGGGATGCGGGTGGCGACGTT
CGCGAACGCGAGGACGGCGCTGGAGGCTCGCCGCGGGATCGGGCGGGCGTTCGCGG
TGGCGTTCAGGTCGGGCGCCGCCATGGGGTTCCTGCTGGCGTCGAGCGCGCTGCTGG
TGCTGTTCGCCGCCGTGAACGCGTTCGGGCTCTACTACGGCGACGACTGGGGGGGGC
TGTACGAGGCGATCACCGGGTACGGGCTCGGGGGGTCGTCCATGGCGCTGTTCGGC
CGCGTCGGCGGCGGGATCTACACCAAGGCGGCCGACGTCGGCGCCGACCTCGTCGG
CAAGGTGGAGCGCAACATCCCCGAGGATGACCCCCGCAACCCCGCGGTAAGCCACG
CCACGTCACTCTCCTCGTCCTCACTCTGTTAAAGATTAAGTTAACACACATAAAAAT
TAAGTTAACGCACATGAAACGAGAAAGCTATTAGCATATAATTAACTTTTCTTTAA
GATAAAGGATTTAAATAAAACCCGGCTTCTACATCCCGAAGAATGTACACAGCC
AACCAAATCCATACAAAAACACTAAGGCTAGTAAACATGATTAACTAAGTTTTA
ATTATTGCTAACTGATAAATTGATATATCTGATAGTTTAAAGCAACTTCTGTATA
AAAAGTTTCTGTAACGTATCGTTTAGTTGTTTAAAAAGCACGCTAATACGAACA
GAGGTAATATCTGAATTTTAATCAGAAAAGAACATGACCGAACCAATTCGATCT
CATATTTTAAAACAACTCAAAGCTGCTTGCATGTTGCATGCGAGCTCGGTTCGA
TCTGAGCCACTGCACTAATCTGAAAGCAAGTCCAGTTGGCCTTTTTTTGTTGCC
TTCCTTAAACATTACTAGGAAGCATGAAACTCACGTGTTCAGTGTTCTCTGCTA
GCTGGCACAGGCGTAACGCATGATTTTTACCGGATTTATTCATAGAAATATAGG
AGAGAGATTGAAAATTTATTGTCTTGTTTGGAGAAAAGGAATTATTTAAAAAGT
CTCGCTAGGAAATATTTAAATTGTGGACAAATTTAAACTGAATTTGACTTAGAA
ATTTAAGTGAATTCAATTCAAACCAACTTTCTTTTTAAAAAGATCGATTGAAACA
AATCTATTTTGGCAGTTGATTTGTGTATGCATCCACTTTGGATTGTTCTTTTGAC
TGAATAGCTCAATATCTGAATATTTGTAATGATTATTTGAGGTTACAAACGAAT
GAACTAAATATTGTTTTCTTTTCGTTTCGTATTATAAGATATTTTGGGTATAATT
TGAATAGATTTATGCATGAATTCAGATACGTACAATATGTATATGTGCGCAAAT
TTATATAGATATTAGTGAATTTAGATGATGTATGAAGAGGCCAAAACGTTGTAT
AACATATAACGGAGAAAATATAGAGTTGAGATCGATTTTGGTTTGAAATAGTTT
TCTCGAAAGAAATTATATATACAAGAAGATTTTATAAAATGCTTTTATATAATTG
TATCGTTTAAAAAACCGTTTAAAAGTTTGGGAAGCTTACAAATTCACCCCCATCG
TTCCCTTCCTTCAAAGAGAAGTGATACATCATATATGGTTTTGTACGTGCGTGTGGAT
ATGATGTGCGCAGGTGATCGCCGACAACGTGGGCGACAACGTGGGCGACATCGCCG
GGATGGGGTCCGACCTGTTCGGGTCGTACGCGGAGTCGTCGTGCGCGGCGCTGTTCG
TGGCGTCCATCTCCTCCTTCGGCGCCGACCACGACTTCGCGGCGATGATGTACCCGC
TGCTGGTGAGCGCCGCCGGCATCGTGGCCTGCGCCGCCACCACGCTCGTCGCCACCG
ACGCCGGCGAGCTCGGCGCCGCCGACGAGGTCGCGCCCGCGCTCAAGCGCCAGATC
CTCATCTCCACCGTGCTCATGACCGCCGCCGTCGCCGCCGTCACCTTCCTCTCCCTCC
CCCGCTCCTTCACCCTCTTCGACTTCGGCGAACGCAAGCTCGTCAAAAATTGGTAAA
AGAAAAAAAAACCTTACATAGTGTGTAATTTATTTTTTTTGGTTTTTTTAAAAAATTA
TAATTACACGGCAGACATACGCACACAAACAAGACCACACTCACATCCCTGTATAC
AATACATTTTTTTTAAAAGTTTTACAATTACACGGGAGACACACGTGCACAGATAAT
ACCACACTCACATCCCTGTCTATGTGCATTCAACAACACATTCTTGATTTCGGAGAA
ATCATGTTAAAAACCTAGTTACAGTGCGTAATATTTATAAGCACCAAGATTTGAATC
TTGTTGGGTGGAGCCATGCACCTATGACTCTAGCAGTTGTACTTGCACCAAATTTGA
TTAAATTATGGAATAATAATAGTTTTGTCTGAATTTTTGTTGAATAATTCAGGCACC
TGTTCATCTGCGTCTCAGCTGGTCTGTGGGGGGATTGGTGATAGGCTACGTCACC
GAGTACTTCACGAGCAATGCTTACGGGTGAAAACGAATTCTACCCACCCAAATTC
GCGATGATTCTACATTTTGTAAAATTTCAAATTTTGATGAAATTTGCGTTCAAATTTC
AAATTGATAGGCCGGTGCAGACGGTGGCGCAGTCGTGCCGGACGGGGGCGGCGACG
AACGTGATCTTCGGCCTCGCCGTGGGGTACAAGTCGGTGATCGTGCCGATCTTCGCC
ATCGCCGGCGCCATCTACGCCAGCTTCCGGCTCGCCGCCATGTACGGCATCGCGCTG
GCTGCGCTGGGGATGCTGAGCACCATCGCCACGGGGCTCACCATCGACGCCTATGG
CCCCATCAGCGACAACGCCGGCGGCATCGCGGAGATGGCCGGCATGCCGCGGCGCG
TGCGCGAGCGCACGGACGCGCTCGACGCCGCCGGGAACACGACGGCGGCGATCGG
GAAGGGGTTCGCGATCGGGTCGGCGGCGCTGGTGTCGCTGGCGCTGTTCGGCGCGT
ACGTGAGCCGGGCGGGGATCCGGACGGTGAACGTGGTGAGCCCCAGGGTGTTCGTC
GGGCTCCTCGCCGGCGCCATGCTCCCCTACTGGTTCTCGGCGATGACGATGCGGAGC
GTGGGGAGCGCGGCGCTGCGGATGGTGGAGGAGGTGCGGCGCCAGTTCGACGAGAT
CCCGGGGCTCGCCGAGGGGCTCGCCGCGCCGGACTACGCCACCTGCGTGAGGATCT
CCACCGACGCGTCGCTGCGGGAGATGGTGGCGCCGGGGGCGCTGGTGATGGCGAGC
CCGCTCGTCGCCGGGACGCTGTTCGGGGTGGAGGCGCTGGCGGGGCTGCTCGCCGG
CGCGCTGGTGTCCGGGGTGCAGGTGGCGATCTCGGCGTCGAACAGCGGCGGCGCGT
GGGACAACGCCAAGAAGTACATCGAGGCCGGGGCGACGGAGGAGGCGAGGTCGCT
GGGGCCCAAGGGCTCCGAGGCGCACAAGGCGGCGGTGATCGGGGACACCATCGGC
GACCCGCTCAAGGACACCTCGGGGCCATCGCTCAACATCCTCGTCAAGCTCATGGCC
GTCGAGGCGCTCGTCTTCGCCCCCTTCTTCGCCGCGCATGGCGGCATCGTCTTCAAC
CACCTCTGATGAAGCTGAGCTCCATTGCTAATAAATTAAGCTGCATGCATGCTCTCG
CCATGATCGATCAGCTGCACCTGCATGCACGCATGGATAAACAAGAAGAAGACGAT
GAGCATTAGGGTTCCTTAACCAGTTGAAACCGATAAGAGTAATAATTGTAGCTCGGG
ATAATGCAGTGTCAGATGTAATCCAAAGTGTCTTGTTGCTTGGGATATGTAATGCGG
TTTGAGACTT
```

### REFERENCES

Ali F, Waters D L L, Ovenden B, Bundock P, Raymond C A, Rose T J (2019) Heat stress during grain fill reduces head rice yield through genotype dependent increased husk biomass and grain breakage. J. Cereal Sci. 90:102820.
Ambardekar A A, Siebenmorgen T J, Counce P A, Lanning S B, Mauromoustakos A (2011) Impact of field-scale nighttime air temperatures during kernel development on rice milling quality. FieldCrop Res. 122:179-185, 10.1016/j.fcr.2011.03.012
Bao J (2014) Genes and QTLs for rice grain quality improvement. In: Rice—Germplasm, Genetics and Improvement (Yan W, Bao J, eds.), Intech Open, London.
Cao Y Y, Chen Y H, Chen M X, Wang Z Q, Wu C F, Bian X C, Yang J C, Zhang J H (2016) Growth characteristics and endosperm structure of superior and inferior spikelets of indica rice under high temperature stress. Biol. Plant. 60:1-11.
Chandran A K N, Sandhu J, Irvin L, Paul P, Dhatt B K, Hussain W, Gao T, Staswick P, Yu H, Morota G, Walia H (2022) Rice Chalky Grain 5 regulates natural variation for grain quality under heat stress. Front. Plant Sci. 13:1026472. doi: 10.3389/fpls.2022.1026472.
Chow C N, Lee T Y, Hung Y C, Li G Z, Tseng K C, Liu Y H, Kuo P L, Zheng H Q, Chang W C (2019) PlantPAN3.0: a new and updated resource for reconstructing transcriptional regulatory networks from ChIP-seq experiments in plants. Nucl. Acids Res. 47 (D1): D1155-D1163. doi: 10.1093/nar/gkyl081.
Counce P A, Bryant R J, Bergman C J, Bautista R C, Wang Y, Siebenmorgen T J, MoldenhauerKAK, Meullenet J C (2005) Rice milling quality grain dimensions, and starch branching as affected by high night temperatures. Cereal Chem. 82:645-648
Custodio M, Cuevas R, Ynion J, Laborte A, Velasco M, Demont M (2019) Rice quality: How is it defined by consumers, industry, food scientists and geneticists? Trends in FoodSci. Tech. 92:122-137. DOI: 10.1016/j.tifs.2019.07.039
Del Rosario A R, Briones V P, Vidal A J (1968) Composition and endosperm structure of developing and mature rice kernel. Cereal Chem. 45:225-235.
Dhatt B K, Abshire N, Paul P, Hasanthika K, Sandhu J, Zhang Q, Obata T, Walia H (2019) Metabolic dynamics of developing rice seeds under high night-time temperature stress. Front. Plant Sci. 10:1443. doi: 10.3389/fpls.2019.01443
Dwiningsih Y, Kumar A, Thomas J, Ruiz C, Alkahtani J, Hashimi A, Pereira A (2021) Identification of genomic regions controlling chalkiness and grain characteristics in aiecombinant inbred line rice population based on high-throughput SNP markers. Genes. 12:1690. 10.3390/genes12111690.
Edwards J D, Jackson A K, McClung A M (2017) Genetic architecture of grain chalk in rice and interactions with a low phytic acid locus. Field Crops Res. 205:116-123.
Esguerra M Q, Hemphill C C, Counce P A (2019). Differential response of Arkansas rice varieties on high nighttime temperature (HNT). Treatments at different reproductive stages. BR Wells Arkansas Rice Research Studies 2019:37-42. Arkansas Agricultural Experiment Station, Research Series 667. Fayetteville, AR: University of Arkansas System, Division of Agriculture.
Esguerra M Q, Hemphill C C, Counce P A (2021) Screening for high night temperature tolerance of popular Arkansas varieties and advanced lines. B.R. Wells Arkansas Rice Research Studies 2020:41-46. Arkansas Agricultural Experiment Station Research Series 676. Fayetteville, AR: University of Arkansas System, Division of Agriculture.
Ferjani A, Segami S, Horiguchi G, Muto Y, Maeshima M, Tsukaya H (2011) Keep an eye on PPi: the vacuolar-type H+-pyrophosphatase regulates post germinative development in Arabidopsis. The Plant Cell 23:2895-2908.
Fitzgerald M A, McCouch S R, Hall R D (2009) Not just a grain of rice: the quest for quality. Trends Plant Sci. 14:133-139.
Franco-Zorrilla J M, López-Vidriero I, Carrasco J L, Godoy M, Vera P, Solano R (2014) DNA binding specificities of plant transcription factors and their potential to define target genes. Proc. Natl. Acad. Sci. USA 111:2367-2372.
Fu Y, Gu Q, Dong Q, Zhang Z, Lin C, Hu W, Pan R, Guan Y, Hu J (2019) Spermidine enhances heat tolerance of rice seeds by modulating endogenous starch and polyamine metabolism. Molecules 24:1395. doi: 10.3390/molecules24071395.
Gann P J, Esguerra M, Counce P A, Srivastava V (2021). Genotype-dependent and heat induced grain chalkiness in rice correlates with the expression patterns of starch biosynthesis genes. Plant-Environ. Inter. 2:165-176.
Gao Y, Liu C, Li Y et al. (2016) QTL analysis for chalkiness of rice and fine mapping of a candidate gene for qACE9. Rice 9, Article number: 41.
Guo T, Liu X, Wan X, Weng J, Liu S, Liu X, Chen M, Li J, Su N, Wu F, Cheng Z, Guo X, Lei C, Wang J, Jiang L, Wan J (2011) Identification of a stable quantitative trait locus for percentage grains with white chalkiness in rice (Oryza sativa). J. Integr. Plant Biol. 53:598-607.
He P, Li S G, Qian Q, Ma Y Q, Li J Z, Wang W M, et al (1999) Genetic analysis of rice grain quality. Theor. Appl. Genet. 98:502-508.
Impa, SM, Raju, B, Hein, NT, Sandhu J, Vara Prasad P V, Walia H, Krishna Jagadish S V (2021) High night temperature effects on wheat and rice: Current status and way forward. Plant Cell Environ. 2021; 44:2049-2065.
Jacobs T B, LaFayette P R, Schmitz R J, Parrott W A (2015) Targeted genome modifications in soybean with CRISPR/Cas9. BMC Biotechnol. 15:16. doi: 10.1186/s12896-015-0131-2
Keeling P L, Myers A M (2010) Biochemistry and genetics of starch synthesis. Annu. Rev. Food Sci. Technol. 1.1:271-303.
Kharabian-Masouleh A, Waters D L, Reinke R F, Ward R, Henry R J (2012) SNP in starch biosynthesis genes associated with nutritional and functional properties of rice. Sci. Rep. 2:557.doi: 10.1038/srep00557
Kobayashi A, Sonoda J, Sugimoto K, Kondo M, Iwasawa N, Hayashi T, Tomita K, Yano M, Shimizu T (2013) Detection and verification of QTLs associated with heat-induced quality decline of rice (Oryza sativa L.) using recombinant inbred lines and near-isogenic lines. Breed. Sci. 63:339-346. doi: 10.1270/jsbbs.63.339.
Lanning S B, Siebenmorgen T J, Counce P A, Ambardekar A A, Mauromoustakos A (2011) Extreme nighttime air temperatures in 2010 impact rice chalkiness and milling quality. Field Crop Res.124:132-136. 10.1016/j.fcr.2011.06.012
Li Y, Fan C, Xing Y, Yun P, Luo L, Yan B, Peng B, Xie W, Wang G, Li X, Xiao J, Xu C, HeY (2014) Chalk5 encodes a vacuolar H (+)-translocating pyrophosphatase influencing grain chalkiness in rice. Nature Genet. 46:398-404.
Lisle A J, Martin M, Fitzgerald M A (2000) Chalky and translucent rice grains differ in starch composition and structure and cooking properties. Cereal Chem. 77:627-632.
Liu Q, Zhang Q, Burton R A, Shirley N J, Atwell B J (2010) Expression of vacuolar H+-pyrophosphatase (OVP3) is under control of an anoxia-inducible promoter in rice. Plant Mol Biol. 72:47-60. doi: 10.1007/s11103-009-9549-z
Lu G, Casaretto J A, Ying S et al. (2017) Overexpression of OsGATA12 regulates chlorophyll content, delays plant senescence and improves rice yield under high density planting. Plant Mol Biol. 94:215-227.
Lunn J E, Feil R, Hendriks J H M, Gibon Y, Morcuende R, Osuna D, Scheible W-R, Carillo P, Hajirezaei M-R, Stitt M (2006) Sugar-induced increases in trehalose 6-phosphate are correlated with redox activation of ADP glucose pyrophosphorylase and higher rates of starch synthesis in Arabidopsis thaliana. Biochem. J. 397:139.
Lyman N B, Jagadish K S V, Nalley L L, Dixon B L, Siebenmorgen T (2013) Neglecting rice milling yield and quality underestimates economic losses from high-temperature stress. PLoS ONE 8: e72157.
Moldenhauer K, Counce P, Hardke J (2018). Rice growth and development. Rice Production Handbook. 192:7-14.
Morita S, Wada H, Matsue Y (2016) Countermeasures for heat damage in rice grain quality under climate change. Plant Prod. Sci. 19:1-11, DOI: 10.1080/1343943X.2015.1128114
Nevame A, Emon R M, Malek M D, Hasan M, Alam M A, Muharam F M, Aslani F, Rafii M, and Ismail M (2018) Relationship between high temperature and formation of chalkiness and their effects on quality of rice. BioMed Res. Inter. 2018:1653721.
Nyrén P, Nore B F, Strid A (1991) Proton-pumping N,N′-dicyclohexylcarbodiimide-sensitive inorganic pyrophosphate synthase from Rhodospirillum rubrum: purification, characterization, and reconstitution. Biochemistry 30:2883-2887.
Ogata H, Goto S, Sato K, Fujibuchi W, Bono H, Kanehisa M (1999) KEGG: Kyoto encyclopedia of genes and genomes. Nucl. Acids Res. 27:29-34.
Pathak B, Zhao S, Manoharan M, Srivastava V (2019) Dual-targeting by CRISPR/Cas9 leads to efficient point mutagenesis but only rare, targeted deletions in the rice genome. 3 Biotech 9:158
Patindol J, Wang Y J (2003) Fine structures and physicochemical properties of starches from chalky and translucent rice kernels. J. Agric. Food Chem. 51:2777-2784.
Paul M J, Primavesi L F, Jhurreea D, Zhang Y (2008) Trehalose metabolism and signaling. Annu. Rev. Plant Biol. 59:417-441.
Phan T T T, Ishibashi Y, Miyazaki M, Tran H T, Okamura K, Tanaka S, Nakamura J, Yuasa T, Iwaya-Inoue M (2013) High temperature-induced repression of the rice sucrose transporter (OsSutl) and starch synthesis-related genes in sink and source organs at milky ripening stage causes chalky grains. J. Agro. Crop Sci. 199:178-188.
Pluskal T, Castillo S, Villar-Briones A et al. (2010) MZmine 2: Modular framework for processing, visualizing, and analyzing mass spectrometry-based molecular profile data. BMC Bioinformatics 11:395.
Ponnu J, Wahl V, Schmid M (2011) Trehalose-6-Phosphate: Connecting plant metabolism and development. Front. Plant Sci. 2:70. doi: 10.3389/fpls.2011.00070
Reyes J C, Muro-Pastor M I, Florencio F J (2004) The GATA family of transcription factors in Arabidopsis and rice. Plant Physiol. 134:1718-1732.
Rezaei Cherati S, Anas M, Liu S, Shanmugam S, Pandey K, Angtuaco S, Shelton R, Khalfaoui A N, Alena S V, Porter E, Fite T, Cao H, Green M J, Basnakian A G, Khodakovskaya M V (2022) Comprehensive risk assessment of carbon nanotubes used for agricultural applications. ACS Nano16: 12061-12072. DOI: 10.1021/acsnano.2c02201
Sreenivasulu N, Butardo V M Jr, Misra G, Cuevas R P, Anacleto R, Kavi Kishor P B (2015) Designing climate-resilient rice with ideal grain quality suited for high-temperature stress. J. Exp. Bot 66:1737-1748.
Tabata M, Hirabayashi H, Takeuchi Y, Ando I, Iida Y, Ohsawa R (2007) Mapping of quantitative trait loci for the occurrence of white-back kernels associated with high temperatures during the ripening period of rice (Oryza sativa L.). Breed. Sci. 57:47-52. doi: 10.1270/jsbbs.57.47.
Tan Y, Xing Y, Li J, Yu S, Xu C, Zhang Q (2000) Genetic bases of appearance quality of rice grains in Shanyou 63, an elite rice hybrid. Theor. Appl. Genet. 101:823-829.10.1007/s001220051549.
Tian L I, Qi L H, Ryu O, Yamagishi T, Sasaki H (2006) Effect of high temperature on sucrose content and sucrose cleaving enzyme activity in rice grain during the filling stage. Rice Sci.: China. 13:205-210
Wada H, Hatakeyama Y, Onda Y, Nonami H, Nakashima T, Erra-Balsells R, Morita S, Hiraoka K, Tanaka F, Nakano H (2018) Multiple strategies for heat adaptation to prevent chalkiness in the rice endosperm. J. Exp. Bot. 70:1299-1311.
Wan X Y, Wan J M, Weng J F, Jiang L, Bi J C, Wang C M, Zhai H Q (2005) Stability of QTLs for rice grain dimension and endosperm chalkiness characteristics across eight environments. Theor. Appl. Genet. 110:1334-1346.
Wang Z, Xu Y, Wang J, Yang J, Zhang J (2012) Polyamine and ethylene interactions in grain filling of superior and inferior spikelets of rice. Plant Grow. Regul. 66:215-228.Webb B D (1985) Criteria of rice quality in the United States. In: Rice Chemistry and Technology (ed. B. O. Juliano), pp 403-442, St. Paul, Minn.: AACC Intl.
Xia J, Sinelnikov I V, Han, B, Wishart D S (2015) MetaboAnalyst 3.0-Making metabolomics more meaningful. Nucl. Acids Res. 43 (W1), W251-257.
Xu J, Henry A, Sreenivasulu N (2020) Rice yield formation under high day and night temperatures-A prerequisite to ensure future food security. Plant, Cell & Environ. 43:1595-1608.
Xu Y J, Jian C Q, Li K, Tian Y F, Zhu K Y, Zhang W Y, Wang W L, Wang Z Q, Yang J C (2021) The role of polyamines in regulating amino acid biosynthesis in rice grains. Food Energy Secur. 10: e306. doi: 10.1002/fes3.306.
Yang J C, Cao Y Y, Zhang H, Liu L J, Zhang J H (2008) Involvement of polyamines in the post-anthesis development of inferior and superior spikelets in rice. Planta 228:137-149.doi: 10.1007/s00425-008-0725-1.
Zhang H, Zhang J, Wei P, Zhang B, Gou F, Feng Z, Mao Y, Yang L, Zhang H, Xu N, Zhu J K (2014) The CRISPR/Cas9 system produces specific and homozygous targeted gene editing in rice in one generation. Plant Biotechnol. J. 12:797-807
Zhao L, Lei J, Huang Y, Zhu S, Chen H, Huang R, Peng Z, Tu Q, Shen X, Yan S (2016) Mapping quantitative trait loci for heat tolerance at anthesis in rice using chromosomal segment substitution lines. Breed. Sci. 66:358-366.

1. A genetically engineered Oryza sativa subsp japonica seed, wherein the RNA expression or protein expression of vacuolar proton pyrophosphatase 5 (VPP5) in the developing seed is reduced by at least 50% as compared to a non-engineered control seed.

2. The genetically engineered seed of claim 1, wherein the genetically engineered seed comprises a mutation in the promoter of VPP5 relative to the control seed.

3. The genetically engineered seed of claim 1, wherein the genetically engineered seed comprises a mutation in the GATA element located in the promoter of VPP5 relative to the control seed.

4. The genetically engineered seed of claim 1, wherein the genetically engineered seed comprises a 3-nucleotide deletion in the GATA element located near position −729 of the VPP5 promoter.

5. The genetically engineered seed of claim 1, wherein the rice is a variety selected from the group consisting of Nipponbare, Taggart, LaGrue, Kitaake, or Diamond.

6. The genetically engineered seed of claim 1, wherein the chalkiness of rice produced from growing the seeds is reduced by at least 50% as compared to rice produced from the control seed, and/or wherein the chalkiness of rice produced from growing the seeds is reduced by at least 50% as compared to rice produced from the control seed when the plants are grown under conditions of high nighttime temperatures.

7. (canceled)

8. A plant or plant part thereof produced by growing the genetically engineered seed of claim 1.

9. A tissue culture of regenerable cells or protoplasts produced from the seed of claim 1.

10. A method of downregulating VPP5 in Oryza sativa comprising:
providing an Oryza sativa seed;
inducing the seed to form a callus;
introducing a targeting nucleic acid into the callus, wherein the nucleic acid comprises a sequence complementary to a gene encoding VPP5 or a transcription regulatory region of the gene encoding VPP5;
introducing a nuclease into the callus, wherein the nuclease causes a change in the nucleic acid sequence of the gene encoding VPP5 or the transcription regulatory region of the gene encoding VPP5 that is targeted by the nucleic acid;
propagating the callus, wherein propagation of the callus produces genetically engineered Oryza sativa plants; and
selecting the genetically engineered Oryza sativa plants for reduced VPP5 expression, and optionally wherein the rice is subsp. japonica.

11. The method of claim 10, wherein the Oryza sativa var. japonica variety is selected from the group consisting of Nipponbare, LaGrue, Taggart, Kitaake, and Diamond.

12. The method of claim 10, wherein the nucleic acid and nuclease are introduced into the callus via a means selected from the group consisting of Agrobacterium mediated transformation, lipofection, nucleofection, electroporation, and particle bombardment.

13. The method of claim 10, wherein the change in the transcription regulatory region of the gene encoding VPP5 includes the addition, deletion, or substitution of one or more bases between −1000 and +1 of the translation start site for VPP5.

14. The method of claim 13, wherein the change includes the addition, deletion, or substitution of one or more bases between −800 and −650 of the translation start site for VPP5.

15. The method of claim 13, wherein the change includes the addition, deletion, or substitution of one or more bases between −750 and −690 of the translation start site for VPP5.

16. The method of claim 13, wherein the change disrupts the GATA element in the promoter region of VPP5.

17. The method of claim 10, wherein the nuclease is a CRISPR-based nuclease.

18. The method of claim 17, wherein:
(a) the nuclease is a Cas9 nuclease protein;
(b) the nuclease is introduced on a nucleic acid construct encoding the nuclease; and/or
(c) the targeting nucleic acid is a gRNA.

19. (canceled)

20. (canceled)

21. The method of claim 10, wherein RNA expression or protein expression of VPP5 in developing seed is reduced by more than 50% as compared to in a non-genetically engineered control.

22. The method of claim 21, wherein the reduction in RNA or protein expression is determined by measuring the level of SEQ ID NO. 31 or SEQ ID NO. 32.

23. The method of claim 10, wherein grain chalk is reduced in rice produced from genetically engineered seed or plants by greater than 50% as compared to a non-genetically engineered control, wherein the genetically engineered seed or plants have reduced expression of VPP-5 as compared to the non-genetically engineered control.

24. (canceled)

25. (canceled)


SEQUENCE LISTING (32 sequences)

SEQ ID NO: 1 - DNA, 23 bp, synthetic construct
  source 1..23: mol_type = other DNA
ccaaagatctacatgacccagag

SEQ ID NO: 2 - DNA, 23 bp, synthetic construct
  source 1..23: mol_type = other DNA
ctagacggtgccgtttgtagcgg

SEQ ID NO: 3 - DNA, 20 bp, synthetic construct
  source 1..20: mol_type = other DNA
ccaaagtacatgacccagag

SEQ ID NO: 4 - DNA, 24 bp, synthetic construct
  source 1..24: mol_type = other DNA
ctagacggtgccgtttgatagcgg

SEQ ID NO: 5 - DNA, 24 bp, synthetic construct
  source 1..24: mol_type = other DNA
ctagacggtgccgtttggtagcgg

SEQ ID NO: 6 - DNA, 27 bp, synthetic construct
  source 1..27: mol_type = other DNA
gattctagacggtgccgtttgtagcgg

SEQ ID NO: 7 - DNA, 31 bp, synthetic construct
  source 1..31: mol_type = other DNA
ccgctatcaaacggcccctccaaaaaccttt

SEQ ID NO: 8 - DNA, 20 bp, synthetic construct
  source 1..20: mol_type = other DNA
cacctgttcatctgcgtctc

SEQ ID NO: 9 - DNA, 22 bp, synthetic construct
  source 1..22: mol_type = other DNA
cgtaagcattgctcgtgaagta

SEQ ID NO: 10 - DNA, 22 bp, synthetic construct
  source 1..22: mol_type = other DNA
ggagtatgtgagggtcaatgtg

SEQ ID NO: 11 - DNA, 21 bp, synthetic construct
  source 1..21: mol_type = other DNA
gttgttgttggtgccttcttc

SEQ ID NO: 12 - DNA, 23 bp, synthetic construct
  source 1..23: mol_type = other DNA
cccgagatatccctgagattaga

SEQ ID NO: 13 - DNA, 23 bp, synthetic construct
  source 1..23: mol_type = other DNA
ccttacctttcttgaccttgcta

SEQ ID NO: 14 - DNA, 20 bp, synthetic construct
  source 1..20: mol_type = other DNA
actgaggaagaggtgctttg

SEQ ID NO: 15 - DNA, 22 bp, synthetic construct
  source 1..22: mol_type = other DNA
gaggattgtgtccgaagatgag

SEQ ID NO: 16 - DNA, 22 bp, synthetic construct
  source 1..22: mol_type = other DNA
gcaagaagcagagagaccatta

SEQ ID NO: 17 - DNA, 22 bp, synthetic construct
  source 1..22: mol_type = other DNA
aactgtaccatctggaatcacc

SEQ ID NO: 18 - DNA, 17 bp, synthetic construct
  source 1..17: mol_type = other DNA
gacctgcacgagcacac

SEQ ID NO: 19 - DNA, 21 bp, synthetic construct
  source 1..21: mol_type = other DNA
gtcaagtcggtgcacataaga

SEQ ID NO: 20 - DNA, 20 bp, synthetic construct
  source 1..20: mol_type = other DNA
gagacgtaccgcaagtacaa

SEQ ID NO: 21 - DNA, 20 bp, synthetic construct
  source 1..20: mol_type = other DNA
gacaaggacctcctcgtaga

SEQ ID NO: 22 - DNA, 22 bp, synthetic construct
  source 1..22: mol_type = other DNA
ggtactggaaagaagaagttcg

SEQ ID NO: 23 - DNA, 23 bp, synthetic construct
  source 1..23: mol_type = other DNA
tccggccatgatgagatgagcaa

SEQ ID NO: 24 - DNA, 22 bp, synthetic construct
  source 1..22: mol_type = other DNA
tggtcagtaatcagccagtttg

SEQ ID NO: 25 - DNA, 22 bp, synthetic construct
  source 1..22: mol_type = other DNA
caaatacttgacgaacagaggc

SEQ ID NO: 26 - DNA, 20 bp, synthetic construct
  source 1..20: mol_type = other DNA
aaccgaacgcgtcctaagtt

SEQ ID NO: 27 - DNA, 20 bp, synthetic construct
  source 1..20: mol_type = other DNA
gaggagaagacgagcgagtg

SEQ ID NO: 28 - DNA, 24 bp, synthetic construct
  source 1..24: mol_type = other DNA
ccaaagaagagtagcctttccaag

SEQ ID NO: 29 - DNA, 5108 bp, synthetic construct
  source 1..5108: mol_type = other DNA
aaagcatggcaacacgaaaatataaccatgacaccatcacaagctcacaaagatggaaatgttgttgtatttatcattggcatattgctctaggcccacatatcattctcgacatgcaaaatgttactttccggttcattacaaattaatctttatttctctacggttaaatttcagttcaaacatttaaagtgataattacttgtcgttcaaaccatcaaaatgttgaaagcgagaaaggttcattaccaaagaaatgttgtcttgaggccaaacggggcctaagggcacgtttggatcctgggcaaggctcatcctaacctcgcttagtaaagctacacgtttggaactggtaaaataatctggcttttggttttggggacgagctagctttgcagggcaagaaaattcttgctagccagtgagaaccgatttagctctccttcgacggcaaactccgttcgcgtgaaaatattggaacaagggcgatgcagacttctatgaaattggcgccgtaaaattagcgatgtttttcaatgactcacccaatcttgtcggcctcttcttccagatcttgttgtgaactctcgctcgctcaaattttgttgtaaattgccctctccaccaacccataaatacaacgaacaatcagtctggatctaaaaggaaaaattgatttatggaagaactcagcaacaagaggaactcatctgtagcagctagccattaatccttgccacaagaagcaaaaccatccaaacagaggagctacaacaaggctagcaagttggcaaatagatggtttagtgctgctaggctagctgggcgagcgaaccgaacgcgtcctaagttaatttgtcctattatatctaacctcgacacttgtactgcacaagacactagtgcgctcccttgaacagctttttttatggctttttcactagaagctgtaacaactttggtttcttttggccatctttcctccaattagaacaggattgcatacatcttaacagcaaagagagatgcaaactattaaagtttatcatcacaggaaaacttatgacatttacaactttgaaattgaaatatagcaagataaatcaagtagcctttccaagtttcttttgaccaaagaagagaaaagtgccaaagatctacatgacccagagttgttttattcaaaaaccaccattcgaaatgaaggtagctagatgcatgatgagggaaattttgttgcatatgggtgaaccccaaagcttacctaatggcattgtccgtcagcatttccagtgcgtccaaacctcaatgagacgcaccgtagataacttatgctcatgcgtcagatacacctaatttgaaccgacggtaaaggattctagacggtgccgtttgtagcggctttttcgctacttttcttggtgagatagaagacagtgacaaaataagttgcaaaaacaacaaaaatgtttatacacatgtacagcatgagctctacggtgtccctggagagcacaagctgtcaccaagccataacaaacggtcgaactgatcttgaacactggatccaatgcatggtttgttcagtgctcaaagagaacaccagcaaactttccatttagaccctcacaagccacaaatatgcggaaaaaaatgtcatattcaccctccaaaaagcttatacaaggacttgtttgtaattatcttaaaatacaggtgggtaattgcaaagtgagacatcttcccttctataaagagcaagcttagcttggtgatcaccaccactccacatctcttctccatcaagaatcaatcactcgctcgtcttctcctccattggagctctcgatcgagcttagctttgcgcttcgttcatccatggcgctcatcggcacggtggcggccgaggtgctcatcccgctcgcggcggtgatcggcatcctcttcgccgtgctccagtggtacatggtgtccagggtggccgtcccgccgcacgacggcgtcggcggcgccgggaaggtggagagggagagcgacggcggcgacggcgacggcgacggcgtcgacgacgaggaggacggcgtggactaccggggcgtggaggcgaggtgcgcggagatccagcacgccatctccgtgggcgcgacgtcgttcctgatgacggagtacaagtacctgggcgcgttcatggcggcgttcgcggcggtcatcttcgtctcgctgggctccgtggggcggttctccacgtcgacggagccgtgcccgtacgacgcggcgaggcggtgccgcccggcgctggcgaacgcggcgttcaccgcggcggcgttcctcctcggcgccaccacctcggtggtctccggctacctcgggatgcgggtggcgacgttcgcgaacgcgaggacggcgctggaggctcgccgcgggatcgggcgggcgttcgcggtggcgttcaggtcgggcgccgccatggggttcctgctggcgtcgagcgcgctgctggtgctgttcgccgccgtgaacgcgttcgggctctactacggcgacgactggggcgggctgtacgaggcgatcaccgggtacgggctcggggggtcgtccatggcgctgttcggccgcgtcggcggcgggatctacaccaaggcggccgacgtcggcgccgacctcgtcggcaaggtggagcgcaacatccccgaggatgacccccgcaaccccgcggtaagccacgccacgtcactctcctcgtcctcactctgttaaagattaagttaacacacataaaaattaagttaacgcacatgaaacgagaaagctattagcatataattaactcccatcgttcccttccttcaaagagaagtgatacatcatatatggttttgtacgtgcgtgtggatatgatgtgcgcaggtgatcgccgacaacgtgggcgacaacgtgggcgacatcgccgggatggggtccgacctgttcgggtcgtacgcggagtcgtcgtgcgcggcgctgttcgtggcgtccatctcctccttcggcgccgaccacgacttcgcggcgatgatgtacccgctgctggtgagcgccgccggcatcgtggcctgcgccgccaccacgctcgtcgccaccgacgccggcgagctcggcgccgccgacgaggtcgcgcccgcgctcaagcgccagatcctcatctccaccgtgctcatgaccgccgccgtcgccgccgtcaccttcctctccctcccccgctccttcaccctcttcgacttcggcgaacgcaagctcgtcaaaaattggtaaaagaaaaaaaaaccttacatagtgtgtaatttatttttttttggtttttttaaaaaattataattacacggcagacatacgcacacaaacaagaccacactcacatccctgtatacaatacattttttttaaaagttttacaattacacgggagacacacgtgcacagataataccacactcacatccctgtctatgtgcattcaacaacacattcttgatttcggagaaatcatgttaaaaacctagttacagtgcgtaatatttataagcaccaagatttgaatcttgttgggtggagccatgcacctatgactctagcagttgtacttgcaccaaatttgattaaattatggaataataatagttttgtctgaatttttgttgaataattcaggcacctgttcatctgcgtctcagctggtctgtgggcgggattggtgataggctacgtcaccgagtacttcacgagcaatgcttacgggtgaaaacgaattctacccacccaaattcgcgatgattctacattttgtaaaatttcaaattttgatgaaatttgcgttcaaatttcaaattgataggccggtgcagacggtggcgcagtcgtgccggacgggggcggcgacgaacgtgatcttcggcctcgccgtggggtacaagtcggtgatcgtgccgatcttcgccatcgccggcgccatctacgccagcttccggctcgccgccatgtacggcatcgcgctggctgcgctggggatgctgagcaccatcgccacggggctcaccatcgacgcctatggccccatcagcgacaacgccggcggcatcgcggagatggccggcatgccgcggcgcgtgcgcgagcgcacggacgcgctcgacgccgccgggaacacgacggcggcgatcgggaaggggttcgcgatcgggtcggcggcgctggtgtcgctggcgctgttcggcgcgtacgtgagccgggcggggatccggacggtgaacgtggtgagccccagggtgttcgtcgggctcctcgccggcgccatgctcccctactggttctcggcgatgacgatgcggagcgtggggagcgcggcgctgcggatggtggaggaggtgcggcgccagttcgacgagatcccggggctcgccgaggggctcgccgcgccggactacgccacctgcgtgaggatctccaccgacgcgtcgctgcgggagatggtggcgccgggggcgctggtgatggcgagcccgctcgtcgccgggacgctgttcggggtggaggcgctggcggggctgctcgccggcgcgctggtgtccggggtgcaggtggcgatctcggcgtcgaacagcggcggcgcgtgggacaacgccaagaagtacatcgaggccggggcgacggaggaggcgaggtcgctggggcccaagggctccgaggcgcacaaggcggcggtgatcggggacaccatcggcgacccgctcaaggacacctcggggccatcgctcaacatcctcgtcaagctcatggccgtcgaggcgctcgtcttcgcccccttcttcgccgcgcatggcggcatcgtcttcaaccacctctgatgaagctgagctccattgctaataaattaagctgcatgcatgctctcgccatgatcgatcagctgcacctgcatgcacgcatggataaacaagaagaagacgatgagcattagggttccttaaccagttgaaaccgataagagtaataattgtagctcgggataatgcagtgtcagatgtaatccaaagtgtcttgttgcttgggatatgtaatgcggtttgagactt

SEQ ID NO: 30 - DNA, 6195 bp, synthetic construct
  source 1..6195: mol_type = other DNA
aaagcatggcaacacgaaaatataaccatgacaccatcacaagctcacaaagatggaaatgttgttgtatttatcattggcatattgctctaggcccacatatcattctcgacatgcaaaatgttactttccggttcattacaaattaatctttatttctctacggttaaatttcagttcaaacatttaaagtgataattacttgtcgttcaaaccatcaaaatgttgaaagcgagaaaggttcattaccaaagaaatgttgtcttgaggccaaacggggcctaagggcacgtttggatcctgggcaaggctcatcctaacctcgcttagtaaagctacacgtttggaactggtaaaataatctggcttttggttttggggacgagctagctttgcagggcaagaaaattcttgctagccagtgagaaccgatttagctctccttcgacggcaaactccgttcgcgtgaaaatattggaacaagggcgatgcaaacttctatgaaattggcgccgtaaaattagcgatgtttttcaatgactcacccaatcttgtcggcctcttcttccagatcttgttgtgaactctcgctcgctcaaattttgttgtaaattgccctctccaccaacccataaatacaacgaacaatcagtctggatctaaaaggaaaaattgatttatggaagaactcagcaacaagaggaactcatctgtagcagctagccattaatccttgccacaagaagcaaaaccatccaaacagaggagctacaacaaggctagcaagttggcaaatagatggtttagtgctgctaggctagctgggcgagcgaaccgaacgcgtcctaagttaatttgtcctattatatctaacctcgacacttgtactgcacaagacactagtgcgctcccttgaacagctttttttatggctttttcactagaagctgtaacaactttggtttcttttggccatctttcctccaattagaacaggattgcatacatcttaacagcaaagagagatgcaaactattaaagtttatcatcacaggaaaacttatgacatttacaactttgaaattgaaatatagcaagataaatcaagtagcctttccaagtttcttttgaccaaagaagagaaaagtgccaaagatctacatgacccagagttgttttattcaaaaaccaccattcgaaatgaaggtagctagatgcatgatgagggaaattttgttgcatatgggtgaaccccaaagcttacctaatggcattgtccgtcagcatttccagtgcgtccaaacctcaatgagacgcaccgtagataacttatgctcatgcgtcagatacacctaatttgaaccgacggtaaaggattctagacggtgccgtttgtagcggctttttcgctacttttcttggtgagatagaagacagtgacaaaataagttgcaaaaacaacaaaaatgtttatacacatgtacagcatgagctctacggtgtccctggagagcacaagctgtcaccaagccataacaaacggtcgaactgatcttgaacactggatccaatgcatggtttgttcagtgctcaaagagaacaccagcaaactttccatttagaccctcacaagccacaaatatgcggaaaaaaatgtcatattcaccctccaaaaagcttatacaaggacttgtttgtaattatcttaaaatacaggtgggtaattgcaaagtgagacatcttcccttctataaagagcaagcttagcttggtgatcaccaccactccacatctcttctccatcaagaatcaatcactcgctcgtcttctcctccattggagctctcgatcgagcttagctttgcgcttcgttcatccatggcgctcatcggcacggtggcggccgaggtgctcatcccgctcgcggcggtgatcggcatcctcttcgccgtgctccagtggtacatggtgtccagggtggccgtcccgccgcacgacggcgtcggcggcgccgggaaggtggagagggagagcgacggcggcgacggcgacggcgacggcgtcgacgacgaggaggacggcgtggactaccggggcgtggaggcgaggtgcgcggagatccagcacgccatctccgtgggcgcgacgtcgttcctgatgacggagtacaagtacctgggcgcgttcatggcggcgttcgcggcggtcatcttcgtctcgctgggctccgtggggcggttctccacgtcgacggagccgtgcccgtacgacgcggcgaggcggtgccgcccggcgctggcgaacgcggcgttcaccgcggcggcgttcctcctcggcgccaccacctcggtggtctccggctacctcgggatgcgggtggcgacgttcgcgaacgcgaggacggcgctggaggctcgccgcgggatcgggcgggcgttcgcggtggcgttcaggtcgggcgccgccatggggttcctgctggcgtcgagcgcgctgctggtgctgttcgccgccgtgaacgcgttcgggctctactacggcgacgactggggcgggctgtacgaggcgatcaccgggtacgggctcggggggtcgtccatggcgctgttcggccgcgtcggcggcgggatctacaccaaggcggccgacgtcggcgccgacctcgtcggcaaggtggagcgcaacatccccgaggatgacccccgcaaccccgcggtaagccacgccacgtcactctcctcgtcctcactctgttaaagattaagttaacacacataaaaattaagttaacgcacatgaaacgagaaagctattagcatataattaacttttctttaagataaaggatttaaataaaacccggcttctacatcccgaagaatgtacacagccaaccaaatccatacaaaaacactaaggctagtaaacatgattaactaagttttaattattgctaactgataaattgatatatctgatagtttaaagcaacttctgtataaaaagtttctgtaacgtatcgtttagttgtttaaaaagcacgctaatacgaacagaggtaatatctgaattttaatcagaaaagaacatgaccgaaccaattcgatctcatattttaaaacaactcaaagctgcttgcatgttgcatgcgagctcggttcgatctgagccactgcactaatctgaaagcaagtccagttggcctttttttgttgccttccttaaacattactaggaagcatgaaactcacgtgttcagtgttctctgctagctggcacaggcgtaacgcatgatttttaccggatttattcatagaaatataggagagagattgaaaatttattgtcttgtttggagaaaaggaattatttaaaaagtctcgctaggaaatatttaaattgtggacaaatttaaactgaatttgacttagaaatttaagtgaattcaattcaaaccaactttctttttaaaaagatcgattgaaacaaatctattttggcagttgatttgtgtatgcatccactttggattgttcttttgactgaatagctcaatatctgaatatttgtaatgattatttgaggttacaaacgaatgaactaaatattgttttcttttcgtttcgtattataagatattttgggtataatttgaatagatttatgcatgaattcagatacgtacaatatgtatatgtgcgcaaatttatatagatattagtgaatttagatgatgtatgaagaggccaaaacgttgtataacatataacggagaaaatatagagttgagatcgattttggtttgaaatagttttctcgaaagaaattatatatacaagaagattttataaaatgcttttatataattgtatcgtttaaaaaaccgtttaaaagtttgggaagcttacaaattcacccccatcgttcccttccttcaaagagaagtgatacatcatatatggttttgtacgtgcgtgtggatatgatgtgcgcaggtgatcgccgacaacgtgggcgacaacgtgggcgacatcgccgggatggggtccgacctgttcgggtcgtacgcggagtcgtcgtgcgcggcgctgttcgtggcgtccatctcctccttcggcgccgaccacgacttcgcggcgatgatgtacccgctgctggtgagcgccgccggcatcgtggcctgcgccgccaccacgctcgtcgccaccgacgccggcgagctcggcgccgccgacgaggtcgcgcccgcgctcaagcgccagatcctcatctccaccgtgctcatgaccgccgccgtcgccgccgtcaccttcctctccctcccccgctccttcaccctcttcgacttcggcgaacgcaagctcgtcaaaaattggtaaaagaaaaaaaaaccttacatagtgtgtaatttattttttttggtttttttaaaaaattataattacacggcagacatacgcacacaaacaagaccacactcacatccctgtatacaatacattttttttaaaagttttacaattacacgggagacacacgtgcacagataataccacactcacatccctgtctatgtgcattcaacaacacattcttgatttcggagaaatcatgttaaaaacctagttacagtgcgtaatatttataagcaccaagatttgaatcttgttgggtggagccatgcacctatgactctagcagttgtacttgcaccaaatttgattaaattatggaataataatagttttgtctgaatttttgttgaataattcaggcacctgttcatctgcgtctcagctggtctgtgggcgggattggtgataggctacgtcaccgagtacttcacgagcaatgcttacgggtgaaaacgaattctacccacccaaattcgcgatgattctacattttgtaaaatttcaaattttgatgaaatttgcgttcaaatttcaaattgataggccggtgcagacggtggcgcagtcgtgccggacgggggcggcgacgaacgtgatcttcggcctcgccgtggggtacaagtcggtgatcgtgccgatcttcgccatcgccggcgccatctacgccagcttccggctcgccgccatgtacggcatcgcgctggctgcgctggggatgctgagcaccatcgccacggggctcaccatcgacgcctatggccccatcagcgacaacgccggcggcatcgcggagatggccggcatgccgcggcgcgtgcgcgagcgcacggacgcgctcgacgccgccgggaacacgacggcggcgatcgggaaggggttcgcgatcgggtcggcggcgctggtgtcgctggcgctgttcggcgcgtacgtgagccgggcggggatccggacggtgaacgtggtgagccccagggtgttcgtcgggctcctcgccggcgccatgctcccctactggttctcggcgatgacgatgcggagcgtggggagcgcggcgctgcggatggtggaggaggtgcggcgccagttcgacgagatcccggggctcgccgaggggctcgccgcgccggactacgccacctgcgtgaggatctccaccgacgcgtcgctgcgggagatggtggcgccgggggcgctggtgatggcgagcccgctcgtcgccgggacgctgttcggggtggaggcgctggcggggctgctcgccggcgcgctggtgtccggggtgcaggtggcgatctcggcgtcgaacagcggcggcgcgtgggacaacgccaagaagtacatcgaggccggggcgacggaggaggcgaggtcgctggggcccaagggctccgaggcgcacaaggcggcggtgatcggggacaccatcggcgacccgctcaaggacacctcggggccatcgctcaacatcctcgtcaagctcatggccgtcgaggcgctcgtcttcgcccccttcttcgccgcgcatggcggcatcgtcttcaaccacctctgatgaagctgagctccattgctaataaattaagctgcatgcatgctctcgccatgatcgatcagctgcacctgcatgcacgcatggataaacaagaagaagacgatgagcattagggttccttaaccagttgaaaccgataagagtaataattgtagctcgggataatgcagtgtcagatgtaatccaaagtgtcttgttgcttgggatatgtaatgcggtttgagactt

SEQ ID NO: 31 - protein, 770 aa, synthetic construct
MALIGTVAAEVLIPLAAVIGILFAVLQWYMVSRVAVPPHDGVGGAGKVERESDGGDGDGDGVDDEEDGVDYRGVEARCAEIQHAISVGATSFLMTEYKYLGAFMAAFAAVIFVSLGSVGRFSTSTEPCPYDAARRCRPALANAAFTAAAFLLGATTSVVSGYLGMRVATFANARTALEARRGIGRAFAVAFRSGAAMGFLLASSALLVLFAAVNAFGLYYGDDWGGLYEAITGYGLGGSSMALFGRVGGGIYTKAADVGADLVGKVERNIPEDDPRNPAVIADNVGDNVGDIAGMGSDLFGSYAESSCAALFVASISSFGADHDFAAMMYPLLVSAAGIVACAATTLVATDAGELGAADEVAPALKRQILISTVLMTAAVAAVTFLSLPRSFTLFDFGERKLVKNWHLFICVSAGLWAGLVIGYVTEYFTSNAYGPVQTVAQSCRTGAATNVIFGLAVGYKSVIVPIFAIAGAIYASFRLAAMYGIALAALGMLSTIATGLTIDAYGPISDNAGGIAEMAGMPRRVRERTDALDAAGNTTAAIGKGFAIGSAALVSLALFGAYVSRAGIRTVNVVSPRVFVGLLAGAMLPYWFSAMTMRSVGSAALRMVEEVRRQFDEIPGLAEGLAAPDYATCVRISTDASLREMVAPGALVMASPLVAGTLFGVEALAGLLAGALVSGVQVAISASNSGGAWDNAKKYIEAGATEEARSLGPKGSEAHKAAVIGDTIGDPLKDTSGPSLNILVKLMAVEALVFAPFFAAHGGIVFNHL

SEQ ID NO: 32 - DNA, 2661 bp, synthetic construct
  source 1..2661: mol_type = other DNA
actccacatctcttctccatcaagaatcaatcactcgctcgtcttctcctccattggagctctcgatcgagcttagctttgcgcttcgttcatccatggcgctcatcggcacggtggcggccgaggtgctcatcccgctcgcggcggtgatcggcatcctcttcgccgtgctccagtggtacatggtgtccagggtggccgtcccgccgcacgacggcgtcggcggcgccgggaaggtggagagggagagcgacggcggcgacggcgacggcgacggcgtcgacgacgaggaggacggcgtggactaccggggcgtggaggcgaggtgcgcggagatccagcacgccatctccgtgggcgcgacgtcgttcctgatgacggagtacaagtacctgggcgcgttcatggcggcgttcgcggcggtcatcttcgtctcgctgggctccgtggggcggttctccacgtcgacggagccgtgcccgtacgacgcggcgaggcggtgccgcccggcgctggcgaacgcggcgttcaccgcggcggcgttcctcctcggcgccaccacctcggtggtctccggctacctcgggatgcgggtggcgacgttcgcgaacgcgaggacggcgctggaggctcgccgcgggatcgggcgggcgttcgcggtggcgttcaggtcgggcgccgccatggggttcctgctggcgtcgagcgcgctgctggtgctgttcgccgccgtgaacgcgttcgggctctactacggcgacgactggggcgggctgtacgaggcgatcaccgggtacgggctcggggggtcgtccatggcgctgttcggccgcgtcggcggcgggatctacaccaaggcggccgacgtcggcgccgacctcgtcggcaaggtggagcgcaacatccccgaggatgacccccgcaaccccgcggtgatcgccgacaacgtgggcgacaacgtgggcgacatcgccgggatggggtccgacctgtttgggtcgtacgcggagtcgtcgtgcgcggcgctgttcgtggcgtccatctcctccttcggcgccgaccacgacttcgcggcgatgatgtacccgctgctggtgagcgccgccggcatcgtggcctgcgccgccaccacgctcgtcgccaccgacgccggcgagctcggcgccgccgacgaggtcgcgcccgcgctcaagcgccagatcctcatctccaccgtgctcatgaccgccgccgtcgccgccgtcaccttcctctccctcccccgctccttcaccctcttcgacttcggcgaacgcaagctcgtcaaaaattggcacctgttcatctgcgtctcagctggtctgtgggcgggattggtgataggctacgtcaccgagtacttcacgagcaatgcttacgggccggtgcagacggtggcgcagtcgtgccggacgggggcggcgacgaacgtgatcttcggcctcgccgtggggtacaagtcggtgatcgtgccgatcttcgccatcgccggcgccatctacgccagcttccggctcgccgccatgtacggcatcgcgctggctgcgctggggatgctgagcaccatcgccacggggctcaccatcgacgcctatggccccatcagcgacaacgccggcggcatcgcggagatggccggcatgccgcggcgcgtgcgcgagcgcacggacgcgctcgacgccgccgggaacacgacggcggcgatcgggaaggggttcgcgatcgggtcggcggcgctggtgtcgctggcgctgttcggcgcgtacgtgagccgggcggggatccggacggtgaacgtggtgagccccagggtgttcgtcgggctcctcgccggcgccatgctcccctactggttctcggcgatgacgatgcggagcgtggggagcgcggcgctgcggatggtggaggaggtgcggcgccagttcgacgagatcccggggctcgccgaggggctcgccgcgccggactacgccacctgcgtgaggatctccaccgacgcgtcgctgcgggagatggtggcgccgggggcgctggtgatggcgagcccgctcgtcgccgggacgctgttcggggtggaggcgctggcggggctgctcgccggcgcgctggtgtccggggtgcaggtggcgatctcggcgtcgaacagcggcggcgcgtgggacaacgccaagaagtacatcgaggccggggcgacggaggaggcgaggtcgctggggcccaagggctccgaggcgcacaaggcggcggtgatcggggacaccatcggcgacccgctcaaggacacctcggggccatcgctcaacatcctcgtcaagctcatggccgtcgaggcgctcgtcttcgcccccttcttcgccgcgcatggcggcatcgtcttcaaccacctctgatgaagctgagctccattgctaataaattaagctgcatgcatgctctcgccatgatcgatcagctgcacctgcatgcacgcatggataaacaagaagaagacgatgagcattagggttccttaaccagttgaaaccgataagagtaataattgtagctcgggataatgcagtgtcagatgtaatccaaagtgtcttgttgcttgggatatgtaatgcggtttgagacttaggatgtaatccaatgttgttttag